(19) United States
(12) Patent Application Publication
(10) Pub. No.: US 2023/0151115 A1
(43) Pub. Date: May 18, 2023

(54) MULTISPECIFIC ANTIBODY AND METHOD FOR PRODUCING SAME

(71) Applicant: University Public Corporation Osaka, Osaka-shi, Osaka (JP)

(72) Inventors: Takeshi NAKANISHI, Osaka-shi, Osaka (JP); Masaya KITAMURA, Osaka-shi, Osaka (JP); Taro TACHIBANA, Osaka-shi, Osaka (JP)

(21) Appl. No.: 17/907,659

(22) PCT Filed: Mar. 26, 2021
(86) PCT No.: PCT/JP2021/012911

§ 371 Date: Sep. 28, 2022

(30) Foreign Application Priority Data
Mar. 31, 2020 (JP) 2020-063869

(51) Int. Cl. C07K 16/32; C07K 16/28

(52) U.S. Cl. CPC C07K 16/32; C07K 16/2887; C07K 16/2809; C07K 16/2878; C07K 2317/31; C07K 2317/55; C07K 2317/522; C07K 2317/524; C07K 2317/56; C07K 2317/622; C07K 2317/35; C07K 2317/73; C07K 2317/526

(57) ABSTRACT

The present invention provides a novel antibody format that does not use hetero-association technology and that is theoretically free of by-products having immune activity. This multispecific antibody has a Fab region that includes one polypeptide a chain and two polypeptide b chains. The polypeptide a chain includes a polypeptide in which a variable region Va1, a stationary region Ca1, a peptide linker LL, a variable region Va2 and a stationary region Ca are linked in the stated order. The polypeptide b chain includes a polypeptide in which a variable region Vb is linked to a stationary region Cb, which is linked to the stationary region Ca1 or the stationary region Ca2.

### TECHNICAL FIELD

The present invention relates to a multispecific antibody and a method for producing the multispecific antibody.

### BACKGROUND ART

Bispecific antibodies are artificial antibodies that are highly functionalized to target two different antigens by combining two antibodies. The high functionality of bispecific antibodies leads to characteristic mechanisms of action such as recruitment of immune cells, inhibition of signal transduction via receptors, and mediating association between proteins (Non-Patent Document 1).

Owing to such characteristic mechanisms of action, bispecific antibodies are expected to be a driving force for next-generation antibody drugs. Therefore, many studies have been conducted on bispecific antibodies, and currently, 60 or more kinds of antibody formats have been reported (Non-Patent Document 2).

Bispecific antibodies are basically prepared by introducing two kinds of heavy chain genes and two kinds of light chain genes into an animal cell, expressing four kinds of polypeptide chains, and spontaneously associating the polypeptide chains. However, when the expressed four kinds of polypeptide chains associate to constitute an antibody, each polypeptide chain is randomly selected, and thus an undesirable antibody including a homo-associated antibody is inevitably generated as a by-product. In this case, the yield of a target antibody is theoretically 12.5%.

Therefore, in the preparation of a bispecific antibody, it is an important problem to improve the yield of a desired antibody by suppressing the production of by-products. In order to solve such a problem, research to promote hetero-association of polypeptide chains has been energetically advanced. In such a hetero-association technology, introduction of various mutations has been proposed, such as a method of introducing substitutions into a CH1 domain and a CL domain (Patent Document 1) and a method of introducing mutations into a CH3 domain (Non-Patent Document 3 and Patent Document 2). Owing to accumulation of such a large number of hetero-association technologies, the yield of a bispecific antibody by hetero-association exceeds 90% (Non-Patent Document 4).

On the other hand, it has been pointed out that a by-product generated in a bispecific antibody production process may cause unexpected activation of immune cells and increase the risk of side effects when used as an antibody drug (Non-Patent Document 5).

### PRIOR ART DOCUMENTS

### Non-Patent Documents

Non-Patent Document 1: Drug Discovery Today 20, 838?847 (2015)
Non-Patent Document 2: Molecular Immunology 67, 95?106 (2015)
Non-Patent Document 3: HFront. Immunol. 7, 394 (2016)
Non-Patent Document 4: MAbs, 9, 182-212 (2017)
Non-Patent Document 5: Bispecific Antibody Development Programs: Guidance for Industry, U.S. Food and Drug Administration (2019)

### PATENT DOCUMENTS

Patent Document 1: WO 2006/106905 A
Patent Document 2: WO 2013/157953 A

### SUMMARY OF THE INVENTION

### Problems to be Solved by the Invention

The hetero-association technology that has been repeatedly improved in the production of bispecific antibodies can be said to be an elegant technology in view of greatly contributing to improvement of yield. However, no matter how much the hetero-association technology is improved, theoretically, it is inevitable that a homo-associated antibody is generated as a by-product. The homo-associated antibody causes an undesired immune reaction even when it is several %, and affects an increase in risk of side effects and the like. In order to remove such a homo-associated antibody, a special and complicated separation and purification step is essential. In view of these problems, it has to be said that the method for producing a bispecific antibody based on hetero-association is essentially limited.

Therefore, in the production of bispecific antibodies, the present inventors have changed the conventional paradigm based on the hetero-association technology, and have focused on an approach that is theoretically free of by-products exhibiting immune activity.

That is, an object of the present invention is to provide a novel antibody format that does not use a hetero-association technology and that is theoretically free of by-products exhibiting immune activity.

### Means for Solving the Problem

As a result of intensive studies, the present inventors have found that, when a bispecific antibody is designed such that a constant region in one arm and a variable region in the other arm are linked by a peptide linker so that variable regions of both arms are present in the same polypeptide chain, there are only two kinds of polypeptide chains constituting the bispecific antibody, and a by-product exhibiting immune activity is not theoretically generated. The present invention has been completed based on this finding. That is, the present invention provides inventions of the following aspects.

Item 1. A multispecific antibody having a Fab region that includes one polypeptide a chain below and two polypeptide b chains below:

the polypeptide a chain including a polypeptide in which a variable region Va1, a constant region Ca1, a peptide linker LL, a variable region Va2, and a constant region Ca2 are linked in the stated order; and the polypeptide b chain including a polypeptide in which a variable region Vb is linked to a constant region Cb binding to the constant region Ca1 or the constant region Ca2.

Item 2. The multispecific antibody according to item 1, wherein a length of the peptide linker LL is 70 to 280 Å.

Item 3. The multispecific antibody according to item 1 or 2, wherein the peptide linker LL includes a protease recognition sequence.

Item 4. The multispecific antibody according to item 3, wherein the peptide linker LL includes a protease recognition sequence Lr1 on the constant region Ca1 side and a protease recognition sequence Lr2 on the variable region Va2 side.

Item 5. The multispecific antibody according to any one of items 1 to 4, wherein the multispecific antibody is IgD, IgE, IgG, or F(ab′)₂.

Item 6. The multispecific antibody according to any one of items 1 to 5, wherein the polypeptide a chain includes a polypeptide in which a heavy-chain variable region VHa1, a heavy-chain constant region CHa1, a peptide linker LL, a heavy-chain variable region VHa2, and a heavy-chain constant region CHa2 are linked in the stated order.

Item 7. The multispecific antibody according to any one of items 1 to 5, wherein the polypeptide a chain includes a polypeptide in which a heavy-chain variable region VHa1, a light-chain constant region CLa1, a peptide linker LL, a heavy-chain variable region VHa2, and a light-chain constant region CLa2 are linked in the stated order.

Item 8. The multispecific antibody according to any one of items 1 to 7, wherein a single-chain antibody further binds to the variable region Va1 and/or the constant region Ca2.

Item 9. A multispecific antibody having a Fab region including one polypeptide a′ chain below, one polypeptide a″ chain below, and two polypeptide b chains below:

the polypeptide a′ chain including a polypeptide in which a variable region Va1, a constant region Ca1, and a cleavage fragment Lr1′ of a protease recognition sequence Lr1 are linked in the stated order;

the polypeptide a″ chain including a polypeptide in which a cleavage fragment Lr2′ of a protease recognition sequence Lr2, a variable region Va2, and a constant region Ca2 are linked in the stated order; and

the polypeptide b chain including a polypeptide in which a variable region Vb is linked to a constant region Cb binding to the constant region Ca1 or the constant region Ca2.

Item 10. A DNA encoding a polypeptide a chain including a polypeptide in which a variable region Va1, a constant region Ca1, a peptide linker LL, a variable region Va2, and a constant region Ca2 are linked in the stated order, the DNA being used to prepare the multispecific antibody according to any one of items 1 to 9.

Item 11. A transformant obtained by transforming a host with:

a recombinant vector va that includes a DNA encoding a polypeptide a chain including a polypeptide in which a variable region Va1, a constant region Ca1, a peptide linker LL, a variable region Va2, and a constant region Ca2 are linked in the stated order; and

a recombinant vector vb that includes a DNA encoding a polypeptide b chain including a polypeptide in which a variable region Vb is linked to a constant region Cb binding to the constant region Ca1 or the constant region Ca2.

Item 12. A method for producing a multispecific antibody, including an antibody production step of culturing a transformant, the transformant being obtained by transforming a host with:

a recombinant vector va that includes a DNA encoding a polypeptide a chain including a polypeptide in which a variable region Va1, a constant region Ca1, a peptide linker LL, a variable region Va2, and a constant region Ca2 are linked in the stated order; and

a recombinant vector vb that includes a DNA encoding a polypeptide b chain including a polypeptide in which a variable region Vb is linked to a constant region Cb binding to the constant region Ca1 or the constant region Ca2.

Item 13. The method for producing a multispecific antibody according to item 12, wherein the peptide linker LL includes a protease recognition sequence Lr1 on the constant region Ca1 side and a protease recognition sequence Lr2 on the variable region Va2 side, and

the method further includes a linker cleavage step of cleaving the peptide linker LL in the produced antibody by using proteases corresponding to the protease recognition sequence Lr1 and the protease recognition sequence Lr2 after the antibody production step.

Item 14. A multispecific antibody preparation kit, including:

an expression vector va′ including a cloning site CS1 for incorporating a variable region Va1, a DNA encoding a constant region Ca1, a DNA encoding a peptide linker LL, a cloning site CS2 for incorporating a variable region Va2, and a DNA encoding a constant region Ca2 in the stated order; and

an expression vector vb′ including a cloning site CS for incorporating a variable region Vb, and a DNA encoding a constant region Cb binding to the constant region Ca1 or the constant region Ca2.

Item 15. A diagnostic agent including the multispecific antibody according to any one of items 1 to 9.

Item 16. A pharmaceutical composition including the multispecific antibody according to any one of items 1 to 9.

### Advantages of the Invention

According to the present invention, there is provided a novel antibody format that does not use a hetero-association technology and that is theoretically free of by-products exhibiting immune activity.

### BRIEF DESCRIPTION OF THE DRAWINGS

FIG. 1 schematically shows an example of an IgD, E, G-type bispecific antibody among multispecific antibodies of the present invention.

FIG. 2 schematically shows polypeptide chains constituting the multispecific antibody of FIG. 1.

FIG. 3 schematically shows a specific example of the multispecific antibody of FIG. 1.

FIG. 4 schematically shows an example of an F(ab′)-type bispecific antibody among multispecific antibodies of the present invention.

FIG. 5 schematically shows another specific example of the multispecific antibody of FIG. 1.

FIG. 6 schematically shows a by-product generated in the production of the multispecific antibody of FIG. 5.

FIG. 7 schematically shows still another specific example of the multispecific antibody of FIG. 1.

FIG. 8 schematically shows an example of a trispecific antibody among multispecific antibodies of the present invention.

FIG. 9 schematically shows another example of a trispecific antibody among multispecific antibodies of the present invention.

FIG. 10 schematically shows an example of a tetraspecific antibody among multispecific antibodies of the present invention.

FIG. 11 schematically shows an example of a bispecific antibody in which the valence of binding to any epitope is increased among multispecific antibodies of the present invention.

FIG. 12 schematically shows another example of a bispecific antibody in which the valence of binding to any epitope is increased among multispecific antibodies of the present invention.

FIG. 13 schematically shows an example of a trispecific antibody in which the valence of binding to an epitope is increased among multispecific antibodies of the present invention.

FIG. 14 schematically shows another example of an IgD, E, G-type bispecific antibody among multispecific antibodies of the present invention.

FIG. 15 schematically shows a recombinant vector of the present invention.

FIG. 16 schematically shows another example of a multispecific antibody obtained by a method for producing a multispecific antibody of the present invention.

FIG. 17 schematically shows expression vectors included in a kit of the present invention.

FIG. 18 schematically shows an anti-HER2×HER3 bispecific antibody designed and prepared in Test Example 1.

FIG. 19 schematically shows a recombinant vector for expressing a target anti-HER2×HER3 bispecific antibody used in Test Example 1.

FIG. 20 shows an electropherogram under reduction conditions regarding protein A affinity purified products of anti-HER2×HER3 bispecific antibody (Examples 1 to 5) products obtained in Test Example 1.

FIG. 21 shows an electropherogram under non-reduction conditions regarding protein A affinity purified products of anti-HER2×HER3 bispecific antibody (Examples 1 to 5) products obtained in Test Example 1.

FIG. 22 shows a gel filtration chromatogram regarding a protein A affinity purified product of an anti-HER2×HER3 bispecific antibody (Example 1) product obtained in Test Example 1.

FIG. 23 shows an electropherogram under non-reduction conditions regarding a protein A affinity purified product of an anti-HER2×HER3 bispecific antibody (Example 1) product obtained in Test Example 1.

FIG. 24 shows a gel filtration chromatogram regarding protein A affinity purified products of anti-HER2×HER3 bispecific antibody (Examples 2 to 4) products obtained in Test Example 1.

FIG. 25 shows a gel filtration chromatogram regarding a protein A affinity purified product of an anti-HER2×HER3 bispecific antibody (Example 5) product obtained in Test Example 1.

FIG. 26 shows an electropherogram under non-reduction conditions of a fraction obtained by purifying a protein A affinity purified product of an anti-HER2×HER3 bispecific antibody (Example 1) product obtained in Test Example 1 with an IgG-CH1 binding carrier.

FIG. 27 shows a cation exchange chromatogram regarding a protein A affinity purified product of an anti-HER2×HER3 bispecific antibody (Example 1) product obtained in Test Example 1.

FIG. 28 shows results of binding activity evaluation of anti-HER2×HER3 bispecific antibodies (Examples 1 to 4) obtained in Test Example 2 to MCF-7 by flow cytometry.

FIG. 29 shows evaluation results of bispecificity of an anti-HER2×HER3 bispecific antibody (Example 1) obtained in Test Example 2 by an SPR method.

FIG. 30 shows the bispecificity of anti-HER2×HER3 bispecific antibodies (Examples 1 to 5) obtained in Test Example 2 for MCF-7 by results of cell growth inhibitory potency evaluation.

FIG. 31 schematically shows an anti-HER2×HER3 bispecific antibody designed and prepared in Test Example 4.

FIG. 32 schematically shows a recombinant vector for expressing a target anti-HER2×HER3 bispecific antibody used in Test Example 4.

FIG. 33 shows an electropherogram regarding a protein A affinity purified product of an anti-HER2×HER3 bispecific antibody (Example 9) product obtained in Test Example 4.

FIG. 34 shows a gel filtration chromatogram regarding a protein A affinity purified product of an anti-HER2×HER3 bispecific antibody (Example 9) product obtained in Test Example 4.

FIG. 35A schematically shows anti-CD20×CD3 bispecific antibodies (Examples 10 and 11) designed and prepared in Test Example 5.

FIG. 35B schematically shows anti-BCMA×CD3 bispecific antibodies (Examples 12 and 13) designed and prepared in Test Example 5.

FIG. 36A schematically shows a recombinant vector for expressing target anti-CD20×CD3 bispecific antibodies (Examples 10 and 11) used in Test Example 5.

FIG. 36B schematically shows a recombinant vector for expressing target anti-BCMA×CD3 bispecific antibodies (Examples 12 and 13) used in Test Example 5.

FIG. 37 shows a cation exchange chromatogram regarding protein A affinity purified products of anti-CD20×CD3 bispecific antibody (Examples 10 and 11) products and anti-BCMA×CD3 bispecific antibody (Examples 12 and 13) products obtained in Test Example 5.

FIG. 38 shows an electropherogram of peaks separated by cation exchange chromatography of anti-CD20×CD3 bispecific antibody (Examples 10 and 11) products and anti-BCMA×CD3 bispecific antibody (Examples 12 and 13) products obtained in Test Example 5.

FIG. 39 shows flow cytometric analysis results for evaluating the binding activity of anti-CD20×CD3 bispecific antibodies (Examples 10 and 11) obtained in Test Example 6 to CD20-positive cells and CD3-positive cells.

FIG. 40A shows flow cytometric analysis results for evaluating the binding activity of anti-BCMA×CD3 bispecific antibodies (Examples 12 and 13) obtained in Test Example 6 to CD3-positive cells.

FIG. 40B shows analysis results of a surface plasmon resonance method for evaluating the binding activity of anti-BCMA×CD3 bispecific antibodies (Examples 12 and 13) obtained in Test Example 6 to BCMA.

FIG. 41A shows results of a cytotoxicity test using CD20-positive cells and CD3-positive cells for evaluating the bispecificity of an anti-BCMA×CD3 bispecific antibody (Example 10) obtained in Test Example 6.

FIG. 41B shows flow cytometric analysis results for evaluating the bispecificity of anti-BCMA×CD3 bispecific antibodies (Examples 12 and 13) obtained in Test Example 6 for BCMA and CD3-positive cells.

FIG. 42 shows an electropherogram of a linker cleavage product (Example 14) of an anti-HER2×HER3 bispecific antibody of Example 1 obtained in Test Example 7.

FIG. 43 shows flow cytometric analysis results for evaluating the binding activity of a linker cleavage product (Example 14) of an anti-HER2×HER3 bispecific antibody of Example 1 obtained in Test Example 7 to HER2 and HER3 positivity.

### EMBODIMENTS OF THE INVENTION

1. Multispecific Antibody

A multispecific antibody of the present invention has a Fab region including one predetermined polypeptide a chain and two predetermined polypeptide b chains. The predetermined polypeptide a chain includes a polypeptide in which a variable region Va1, a constant region Ca1, a peptide linker LL, a variable region Va2, and a constant region Ca2 are linked in the stated order. The predetermined polypeptide b chain includes a polypeptide in which a variable region Vb is linked to a constant region Cb binding to the constant region Ca1 or the constant region Ca2.

1-1. Multispecific Antibody 1

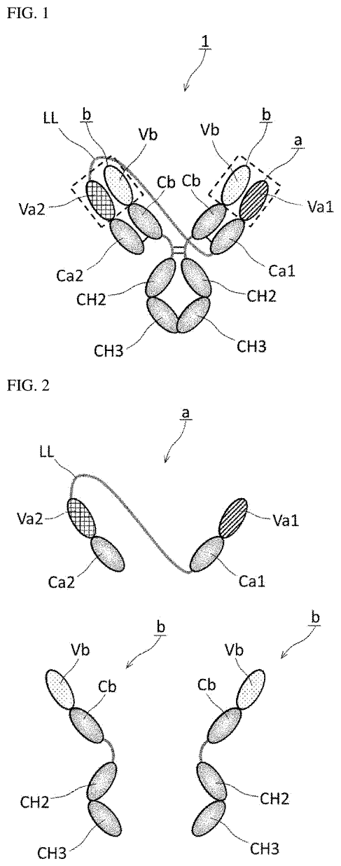

FIG. 1 schematically shows an example of an IgD, E, G-type bispecific antibody (multispecific antibody 1) as a representative of the multispecific antibody of the present invention. In the polypeptide a chain consisting the multispecific antibody 1 of FIG. 1, the variable region Va1, the constant region Ca1, the peptide linker LL, the variable region Va2, and the constant region Ca2 are linked in the stated order. Each of the polypeptide b chains includes the variable region Vb and the constant region Cb, and in this example, further includes a constant region CH2 and a constant region CH3.

FIG. 2 schematically shows polypeptide chains constituting the multispecific antibody 1 of FIG. 1. Usually, the Fab region of an antibody is configured by four polypeptide chains, but in the multispecific antibody 1 of the present invention, of four polypeptide chains constituting a normal Fab region, two polypeptide chains are linked by the peptide linker LL to be unified into the polypeptide a chain, and the remaining two polypeptide chains are shared as the polypeptide b chains. Since the polypeptide chains constituting the antibody are simplified into only two kinds of the polypeptide a chain and the polypeptide b chain, the multispecific antibody 1 of the present invention has a format in which a by-product exhibiting immune activity is not theoretically generated in any combination at the time of association of the polypeptide a chain and the polypeptide b chain.

The multispecific antibody is defined as an antibody having specificity for two or more different epitopes, in other words, an antibody including two or more kinds of variable regions (so-called Fvs) configured by a heavy-chain variable region (so-called VH) and a light-chain variable region (so-called VL). In the multispecific antibody 1 of FIG. 1 that is a bispecific antibody, the variable region Va1 and the variable region Vb, and the variable region Va2 and the variable region Vb constitute two kinds of Fvs (portions surrounded by dashed lines in the drawing) having specificity for different epitopes. As the sequences of the variable region Va1 and the variable region Va2, mutually different sequences may be selected, and as specific sequences of the variable region Va1 and the variable region Va2, Fvs configured by these variable regions are appropriately selected so as to constitute different complementarity determining regions (so-called CDRs). The specific sequence of the CDR is also selected without limitation according to the epitope targeted by the multispecific antibody 1.

Also as the sequences of the constant region CH2 and the constant region CH3, those constituting an Fc region are appropriately selected. The Fc region is a region cleaved by a papain enzyme, and is known as a region related to complement activation, C1q binding and C3 activation, and Fc reporter binding. A specific sequence of the Fc region is selected without limitation according to the isotype (IgD, IgE, or IgG) of the antibody.

The binding between the constant region Cb of the polypeptide b chain and the constant region Ca1 of the polypeptide a chain, the binding between the constant region Cb of the polypeptide b chain and the constant region Ca2 of the polypeptide a chain, and the binding between the two polypeptide b chains are usually disulfide binding.

In the polypeptide a chain, the length of the peptide linker LL linking the constant region Ca1 and the variable region Va2 is not particularly limited, and is, for example, 70 to 280 Å or 20 to 80 amino acid residues.

From the viewpoint of further suppressing the production of by-products, the lower limit of the length of the peptide linker LL is preferably 122 Å or more, more preferably 140 Å or more, further preferably 175 Å or more, even more preferably 210 Å or more, still more preferably 227 Å or more, and particularly preferably 234 Å or more. Alternatively, from the viewpoint of further suppressing the production of by-products, the lower limit of the range of the length of the peptide linker LL is preferably 35 amino acid residues or more, more preferably 40 amino acid residues or more, further preferably 50 amino acid residues or more, even more preferably 60 amino acid residues or more, still more preferably 65 amino acid residues or more, and particularly preferably 67 amino acid residues or more.

The upper limit of the range of the length of the peptide linker LL is preferably 26 Å or less, more preferably 252 Å or less, and further preferably 245 Å or less. In the case of using a multispecific antibody at a low concentration and/or a case where a plurality of epitopes targeted by the multispecific antibody are close to each other, from the viewpoint of enhancing binding activity, the upper limit of the range of the length of the peptide linker LL is preferably 210 Å or less, more preferably 192 Å or less, further preferably 140 Å or less, even more preferably 122 Å or less, still more preferably 105 Å or less, and particularly preferably 87 Å or less. Alternatively, the upper limit of the range of the length of the peptide linker LL is preferably 75 amino acid residues or less, more preferably 72 amino acid residues or less, and further preferably 70 amino acid residues or less. In the case of using a multispecific antibody at a low concentration and/or a case where a plurality of epitopes targeted by the multispecific antibody are close to each other, from the viewpoint of enhancing binding activity, the upper limit of the range of the length of the peptide linker LL is preferably 60 amino acid residues or less, more preferably 55 amino acid residues or less, further preferably 40 amino acid residues or less, even more preferably 35 amino acid residues or less, still more preferably 30 amino acid residues or less, and particularly preferably 25 amino acid residues or less. A specific example of the low concentration is, for example, 2 to 50 nM, preferably 4 to 30 nM, more preferably 6 to 20 nM, and further preferably 8 to 15 nM. Specific examples of the case where a plurality of epitopes targeted by the multispecific antibody are close to each other include a case where a plurality of antigens (for example, two antigens) form a heteromultimer (for example, a heterodimer) on one cell surface.

As a specific sequence of the peptide linker LL, a sequence, which is not accompanied by an undesired bond at the time of expression of the polypeptide a chain or at the time of association between the polypeptide a chain and the polypeptide b chain or does not affect the molecular recognizability of the multispecific antibody 1 itself, is appropriately selected.

The peptide linker LL has, as a main constituent, a basic sequence contributing to linking. The amino acid residue constituting the basic sequence of the peptide linker LL is preferably an amino acid residue having no bulky side chain or having no side chain itself. As such an amino acid residue, a glycine residue, an alanine residue, a serine residue, a threonine residue, an aspartic acid residue, a glutamic acid residue, and the like are preferably exemplified, and a glycine residue and a serine residue are preferably exemplified.

The basic sequence of the peptide linker LL preferably includes a hydrophilic amino acid residue. Examples of such a hydrophilic amino acid residue include a serine residue, a threonine residue, an aspartic acid residue, and a glutamic acid residue, and a serine residue is preferably exemplified. In this case, examples of a hydrophobic amino acid residue other than the hydrophilic amino acid residue constituting the basic sequence of the peptide linker LL include a glycine residue and an alanine residue, and a glycine residue is preferably exemplified. The proportion of the number of hydrophilic amino acid residues in the total number of amino acid residues constituting the peptide linker LL is preferably 10 to 30%, more preferably 15 to 25%, and further preferably 18 to 22%. A preferred sequence of the basic sequence of the peptide linker LL is a sequence in which a hydrophobic amino acid residue and a hydrophilic amino acid residue are alternately repeated, a more preferred sequence is a sequence in which a glycine residue (G) and a serine residue (S) are alternately repeated, and a particularly preferred sequence is a repeat sequence of GGGGS.

1-2. Multispecific Antibody 11

In the multispecific antibody of the present invention, the peptide linker LL may be configured by only the basic sequence (may not include a protease recognition sequence) or may further include a protease recognition sequence in addition to the basic sequence. From the viewpoint of suppressing the production of by-products in the production of the multispecific antibody of the present invention, as the peptide linker LL, one which does not include a protease recognition sequence is preferred. On the other hand, from the viewpoint of performing a process of cleaving the peptide linker LL after producing an antibody, as the peptide linker LL, one which includes a protease recognition sequence is used.

When the peptide linker LL includes a protease recognition sequence, the number of protease recognition sequences per peptide linker LL is not particularly limited, and is, for example, 1 or 2 or more, preferably 1 or 2, and more preferably 2. When the peptide linker LL includes two or more protease recognition sequences, each of the two or more protease recognition sequences may have the same sequence or different sequences. The protease recognition sequence is not particularly limited as long as it is a sequence that is specifically recognized and cleaved by a specific protease.

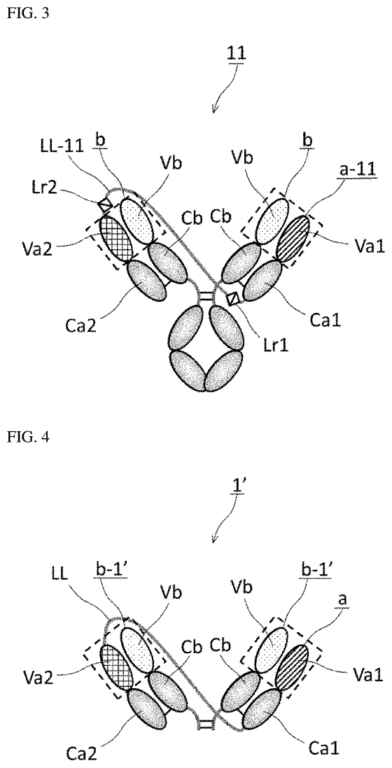

FIG. 3 schematically shows another example of an IgD, E, G-type bispecific antibody (multispecific antibody 11) when the peptide linker LL includes a protease recognition sequence in the multispecific antibody of the present invention. The multispecific antibody 11 of FIG. 3 is the same as the multispecific antibody 1 of FIG. 1, except that the peptide linker L in the multispecific antibody of FIG. 1 is replaced by a peptide linker LL-11 including two protease recognition sequences Lr1 and Lr2, and the polypeptide a chain in the multispecific antibody of FIG. 1 is replaced by a polypeptide a-11 chain including the peptide linker LL-11.

The peptide linker LL-11 in the multispecific antibody 11 specifically includes a protease recognition sequence Lr1 on the constant region Ca1 side and a protease recognition sequence Lr2 on the variable region Va2 side. The protease recognition sequence Lr1 is preferably arranged at a position as close as possible to the constant region Ca1, and more preferably arranged adjacent to the constant region Ca1. The protease recognition sequence Lr2 is preferably arranged at a position as close as possible to the variable region Va2, and more preferably arranged adjacent to the variable region Va2.

1-3. Multispecific Antibody 1′

In the present invention, the shape of the other part of the multispecific antibody is not particularly limited as long as the multispecific antibody has a Fab region including one predetermined polypeptide a chain and two predetermined polypeptide b chains. For example, the Fc region of the antibody is not necessarily required.

FIG. 4 schematically shows an example of an F(ab′)-type bispecific antibody (multispecific antibody 1′) among multispecific antibodies of the present invention. The multispecific antibody 1′ shown in FIG. 4 is the same as the multispecific antibody 1 shown in FIG. 1, except that the polypeptide b chain does not have CH2 and CH3 in the constant region.

1-4. Multispecific Antibody 12

In the above-described multispecific antibodies 1, 11, and 1′, the specific sequences of respective regions of the variable region Va1, the constant region Ca1, the variable region Va2, and the constant region Ca2 of the polypeptide a chain and the polypeptide a-11 chain and the variable region Vb and the constant region Cb of the polypeptide b chain may each independently be a sequence in either the heavy chain or the light chain.

FIG. 5 schematically shows a specific example of an IgD, E, G-type bispecific antibody (multispecific antibody 12) of the multispecific antibody of the present invention. The multispecific antibody 12 of FIG. 5 is an example of the multispecific antibody 1 of FIG. 1, and specifically, a case where the polypeptide a chain and the polypeptide b chain in the multispecific antibody 1 of FIG. 1 are a polypeptide a-12 chain and a polypeptide b-12 chain, respectively, is exemplified.

In this polypeptide a-12 chain, all of the variable region Va1, the constant region Ca1, the variable region Va2, and the constant region Ca2 of the polypeptide a chain in the multispecific antibody 1 of FIG. 1 have a heavy-chain sequence, that is, as shown in FIG. 5, a heavy-chain variable region VHa1, a heavy-chain constant region CHa1, a peptide linker LL, a heavy-chain variable region VHa2, and a heavy-chain constant region CHa2 are linked in the stated order.

In the polypeptide b-12 chain, both the variable region Vb and the constant region Cb of the polypeptide b chain in the multispecific antibody 1 of FIG. 1 have a light-chain sequence, that is, as shown in FIG. 5, a light-chain variable region VLb and a light-chain constant region CLb are linked. The polypeptide b-12 chain has an unnatural domain linked form in which the light-chain constant region CLb is further linked to the constant region CH2 and the constant region CH3. A method for determining the amino acid sequence of such an unnatural domain linking site is known, and those skilled in the art can appropriately determine the amino acid sequence of the linking site in designing of the multispecific antibody 12.

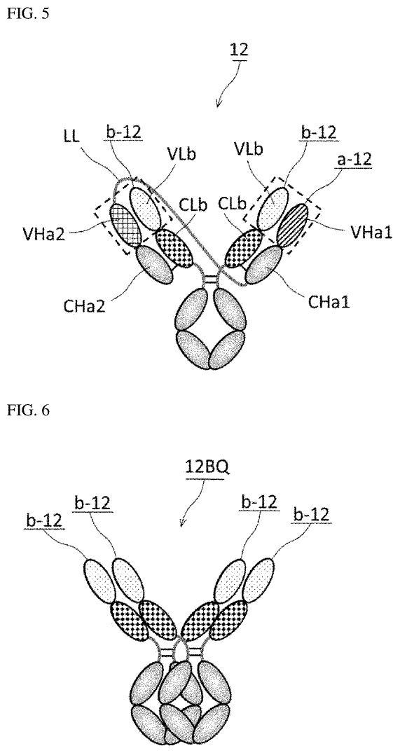

As described above, the multispecific antibody of the present invention theoretically does not produce a by-product exhibiting immune activity. For example, when the polypeptide a-12 chain and the polypeptide b-12 chain constituting the multispecific antibody 12 shown in FIG. 5 associate with each other, a product exhibiting immune activity generated in any combination of these polypeptide chains is theoretically only the multispecific antibody 12. On the other hand, examples of the by-product generated when the polypeptide a-12 chain and the polypeptide b-12 chain constituting the multispecific antibody 12 associate with each other include a tetramer 12BQ of the polypeptide b-12 chain shown in FIG. 6. This by-product is generated with the multispecific antibody 12, but does not exhibit immune activity. Therefore, according to the present invention, unlike conventional multispecific antibodies that inevitably generate a by-product exhibiting immune activity, a special and complicated separation step is not required, and an undesirable immune reaction caused by a slight residual by-product after the separation step can also be avoided.

As a further modified example of the multispecific antibody 12, the peptide linker LL may be replaced with the peptide linker LL-11 including a protease recognition sequence, or may be an F(ab′) type where the Fc region is absent.

1-5. Multispecific Antibody 13

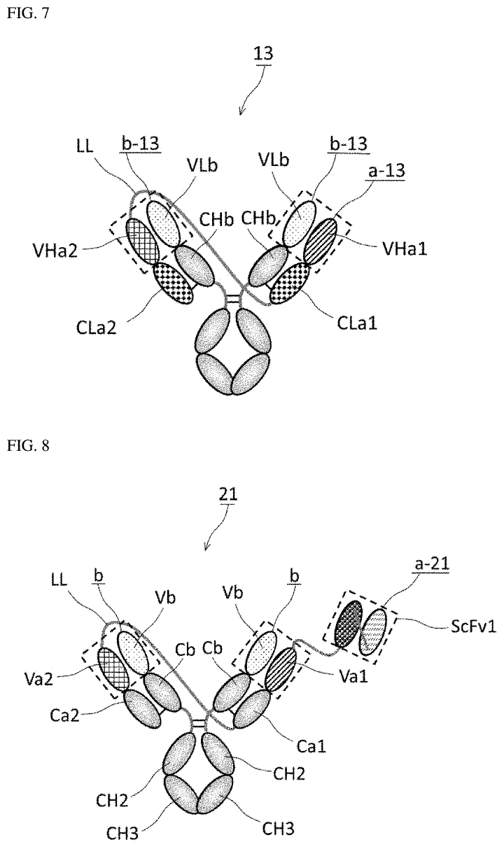

FIG. 7 schematically shows another specific example of an IgD, E, G-type bispecific antibody (multispecific antibody 13) of the multispecific antibody of the present invention. The multispecific antibody 13 of FIG. 7 is another example of the multispecific antibody 1 of FIG. 1, and specifically, a case where the polypeptide a chain and the polypeptide b chain in the multispecific antibody 1 of FIG. 1 are a polypeptide a-13 chain and a polypeptide b-13 chain, respectively, is exemplified.

In this polypeptide a-13 chain, the variable region Va1 and the variable region Va2 of the polypeptide a chain in the multispecific antibody 1 of FIG. 1 have a heavy-chain sequence and the constant region Ca1 and the constant region Ca2 have a light-chain sequence, that is, as shown in FIG. 7, a heavy-chain variable region VHa1, a light-chain constant region CLa1, a peptide linker LL, a heavy-chain variable region VHa2, and a light-chain constant region CLa2 are linked in the stated order.

In the polypeptide b-13 chain, the variable region Vb of the polypeptide b chain in the multispecific antibody 1 of FIG. 1 has a light-chain sequence and the constant region Cb has a heavy-chain sequence, that is, as shown in FIG. 7, a light-chain variable region VLb and a light-chain constant region CHb are linked.

The polypeptide a-13 chain and the polypeptide b-13 chain have an unnatural domain linked form in which a light chain-derived region and a heavy chain-derived region are linked. A method for determining the amino acid sequence of such an unnatural domain linking site is known, and those skilled in the art can appropriately determine the amino acid sequence of the linking site in designing of the multispecific antibody 13.

As described above, also for the multispecific antibody 13, when the polypeptide a-13 chain and the polypeptide b-13 chain associate with each other, a product exhibiting immune activity generated in any combination of these polypeptide chains is theoretically only the multispecific antibody 13. On the other hand, as a by-product, a tetramer corresponding to the tetramer 12BQ in FIG. 6 is not generated. Therefore, the format of the multispecific antibody 13 is still more preferable from the viewpoint of further suppressing the production of by-products and enabling high yield.

As a further modified example of the multispecific antibody 13, the peptide linker LL may be replaced with the peptide linker LL-11 including a protease recognition sequence, or may be an F(ab′) type where the Fc region is absent.

1-6. Multispecific Antibodies 21, 22, and 23

The multispecific antibody of the present invention can be designed such that a single-chain antibody further binds to the variable region Va1 and/or the constant region Ca2.

The single-chain antibody is a well-known structure including a heavy-chain variable region (so-called VH), a light-chain variable region (so-called VL), and a peptide linker linking these variable regions, and constituting a variable region (so-called Fv) configured by VH and VL.

As an example of the multispecific antibody of the present invention to which a single-chain antibody binds, specific examples of trispecific antibodies (multispecific antibodies 21 and 22) and a specific example of a tetraspecific antibody (multispecific antibody 23) are schematically shown in FIGS. 8 to 10.

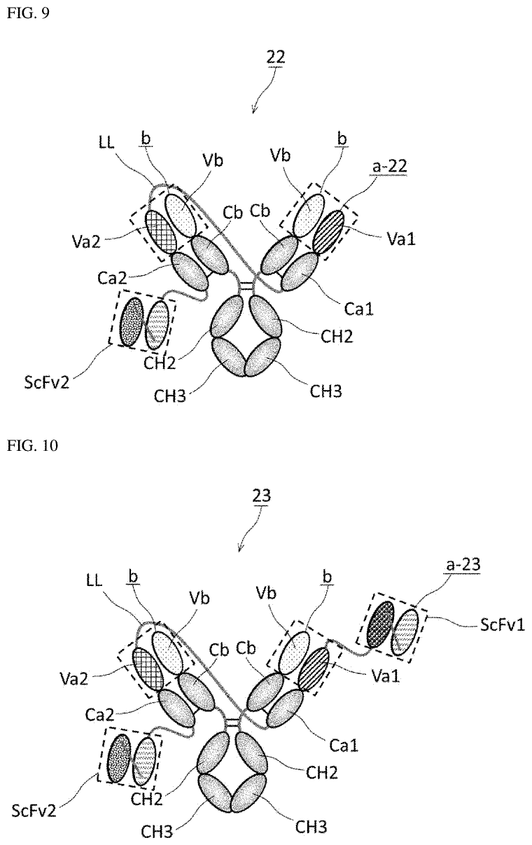

The multispecific antibody 21 shown in FIG. 8 is the same as the multispecific antibody 1 of FIG. 1, except that the polypeptide a chain in the multispecific antibody of FIG. 1 is replaced by a polypeptide a-21 chain in which a single-chain antibody ScFv1 binds to the variable region Va1. The multispecific antibody 22 shown in FIG. 9 is the same as the multispecific antibody 1 of FIG. 1, except that the polypeptide a chain in the multispecific antibody of FIG. 1 is replaced by a polypeptide a-22 chain in which a single-chain antibody ScFv2 binds to the constant region Ca2. The multispecific antibody 23 shown in FIG. 10 is the same as the multispecific antibody 1 of FIG. 1, except that the polypeptide a chain in the multispecific antibody of FIG. 1 is replaced by a polypeptide a-23 chain in which a single-chain antibody ScFv1 binds to the variable region Va1 and a single-chain antibody ScFv2 binds to the constant region Ca2.

In the multispecific antibodies 21, 22, and 23, Fvs configured by VH and VL are indicated by being surrounded by dashed lines, and these Fvs are all configured to have specificity for different epitopes. Therefore, in the multispecific antibodies 21 and 22, the specific sequences of VH and VL of the single-chain antibodies ScFv1 and ScFv2 are appropriately selected so that the complementarity determining region (so-called CDR) of Fv constituted by these VH and VL is different from both of the CDR of Fv configured by the variable region Va1 and the variable region Vb and the CDR of Fv configured by the variable region Va2 and the variable region Vb. In the multispecific antibody 23, VH and VL of the single-chain antibody ScFv1 and VH and VL of the single-chain antibody ScFv2 are selected so as to constitute mutually different CDRs.

In the polypeptide a-21 chain of the multispecific antibody 21, either VH or VL of the single-chain antibody ScFv1 may be linked to the variable region Va1. Similarly, in the polypeptide a-22 chain of the multispecific antibody 22, either VH or VL of the single-chain antibody ScFv2 may be linked to the constant region Ca2. Similarly, in the polypeptide a-23 chain of the multispecific antibody 23, either VH or VL of the single-chain antibody ScFv1 may be linked to the variable region Va1, or either VH or VL of the independent single-chain antibody ScFv2 may be linked to the constant region Ca2.

The sequence and length of the peptide linker linking VH and VL constituting the single-chain antibodies ScFv1 and ScFv2 are appropriately selected by those skilled in the art in consideration of the stability, steric structure formation, antigen recognition property, and the like of the single-chain antibody ScFv. Specific constituent amino acids and sequences are the same as the basic sequence described in the peptide linker LL, and more specific examples thereof include GGGGS or a repeat sequence thereof. Specific examples of the length include about 15 amino acid residues.

The form of linking the single-chain antibodies ScFv1 and ScFv2 to the variable region Va1 or the constant region Ca2 is not particularly limited, and is preferably linking by a peptide linker. The sequence and length of the peptide linker linking the single-chain antibodies ScFv1 and ScFv2 to the variable region Va1 or the constant region Ca2 are appropriately selected by those skilled in the art in consideration of the size and shape of the antigen, the positional relationship between different epitopes, and the like. Specific constituent amino acids and sequences are the same as the basic sequence described in the peptide linker LL, and more specific examples thereof include GGGGS or a repeat sequence thereof. A specific length is, for example, 5 to 20 amino acid residues, preferably 8 to 15 amino acid residues, and more preferably about 10 to 12 amino acid residues.

The specific sequences of the variable region Va1, the constant region Ca1, the variable region Va2, and the constant region Ca2 in the polypeptide a-21 chain, the polypeptide a-22 chain, and the polypeptide a-23 chain and the variable region Vb and the constant region Cb of the polypeptide b chain constituting each of the multispecific antibodies 21 to 23 may each independently be a sequence in either the heavy chain or the light chain.

Therefore, in examples of the multispecific antibodies 21 to 23, similarly to the multispecific antibody 12 of FIG. 5, all the variable region Va1, the constant region Ca1, the variable region Va2, and the constant region Ca2 in the polypeptide a-21 chain, the polypeptide a-22 chain, and the polypeptide a-23 chain may have a heavy-chain sequence, and both the variable region Vb and the constant region Cb of the polypeptide b chain may have a light-chain sequence. In other examples of the multispecific antibodies 21 to 23, similarly to the multispecific antibody 13 of FIG. 7, the variable region Va1 and the variable region Va2 in the polypeptide a-21 chain, the polypeptide a-22 chain, and the polypeptide a-23 chain may have a heavy-chain sequence, the constant region Ca1 and the constant region Ca2 may have a light-chain sequence, and in the polypeptide b chain, the variable region Vb may have a light-chain sequence and the constant region Cb may have a heavy-chain sequence.

As further modified examples of the multispecific antibodies 21 to 23, the peptide linker LL may be replaced with the peptide linker LL-11 including a protease recognition sequence, or the Fc region may be absent.

1-7. Multispecific Antibodies 31, 32, and 33

The multispecific antibody of the present invention can be designed as a multispecific antibody in which the valence for at least one epitope is increased, in addition to those shown in the above “1-6. Multispecific antibodies 21, 22, and 23”, by designing such that a single-chain antibody further binds to the variable region Va1 and/or the constant region Ca2.

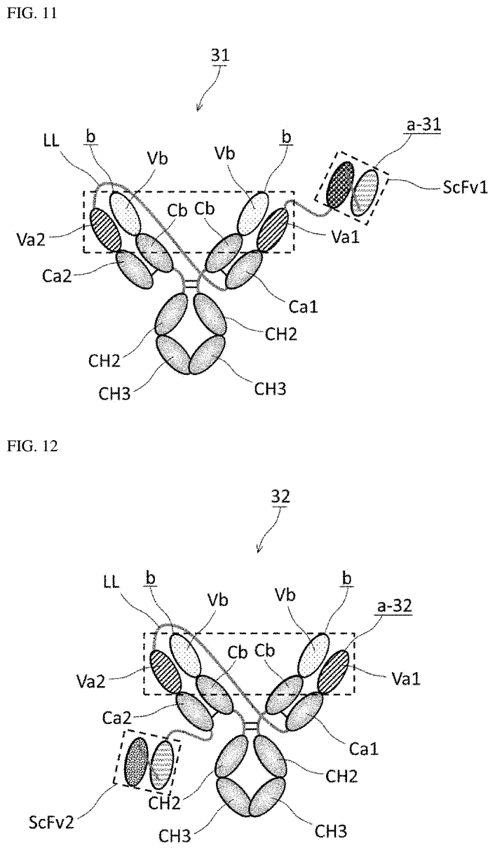

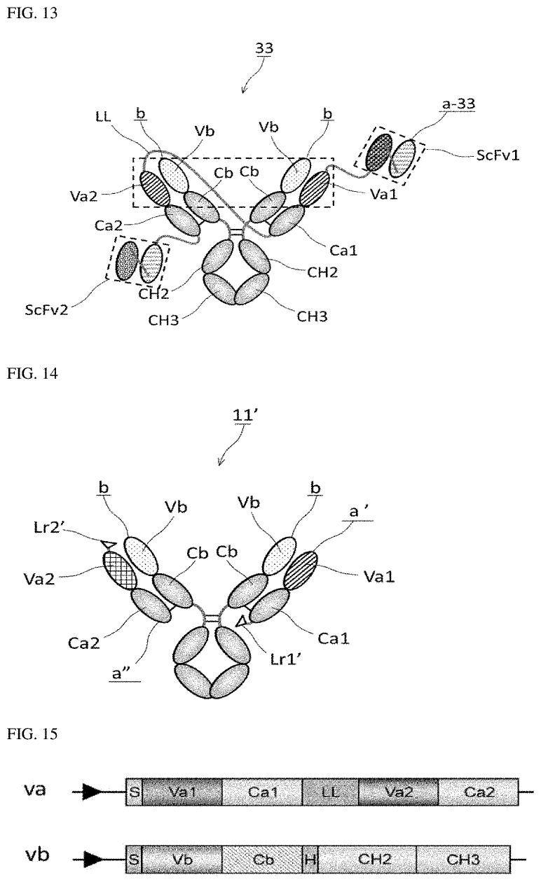

FIGS. 11 and 12 schematically show specific examples of (2+1)-type bispecific antibodies (multispecific antibodies 31 and 32) in the multispecific antibody of the present invention and FIG. 13 schematically shows a specific example of a (2+1+1)-type trispecific antibody (multispecific antibody 33) in the multispecific antibody of the present invention.

The multispecific antibody 31 shown in FIG. 11 is the same as the multispecific antibody 1 of FIG. 1, except that the polypeptide a chain in the multispecific antibody of FIG. 1 is replaced by a polypeptide a-31 chain. The polypeptide a-31 chain is designed such that the single-chain antibody ScFv1 binds to the variable region Va1, and the CDR of Fv including the variable region Va2 and the variable region Vb is common to the CDR of Fv including the variable region Va1 and the variable region Vb and different from the CDR of the single-chain antibody ScFv1.

The multispecific antibody 32 shown in FIG. 12 is the same as the multispecific antibody 1 of FIG. 1, except that the polypeptide a chain in the multispecific antibody of FIG. 1 is replaced by a polypeptide a-32 chain. The polypeptide a-32 chain is designed such that the single-chain antibody ScFv2 binds to the constant region Ca2, and the CDR of Fv including the variable region Va2 and the variable region Vb is common to the CDR of Fv including the variable region Va1 and the variable region Vb and different from the CDR of the single-chain antibody ScFv2.

The multispecific antibody 33 shown in FIG. 13 is the same as the multispecific antibody 1 of FIG. 1, except that the polypeptide a chain in the multispecific antibody of FIG. 1 is replaced by a polypeptide a-33 chain. The polypeptide a-33 chain is designed such that the single-chain antibody ScFv1 binds to the variable region Va1, the single-chain antibody ScFv2 having a CDR different from the single-chain antibody ScFv1 binds to the constant region Ca2, and the CDR of Fv including the variable region Va2 and the variable region Vb is common to the CDR of Fv including the variable region Va1 and the variable region Vb and different from the CDRs of both the single-chain antibody ScFv1 and the single-chain antibody ScFv2.

In the multispecific antibodies 31, 32, and 33 of FIGS. 11 to 13, each Fv having specificity for a specific epitope is surrounded by dashed lines. For example, in the multispecific antibodies 31 and 32, among three Fvs in total, two Fvs in both arms of the antibody are shared, and Fv corresponding to an epitope different from this Fv is carried by the single-chain antibody ScFv1 or the single-chain antibody ScFv2. That is, the multispecific antibodies 31 and 32 are designed to have an increased binding titer to one of target epitopes while being bispecific antibodies. In the multispecific antibody 33, among four Fvs in total, two Fvs in both arms of the antibody are shared, and Fvs corresponding to two kinds of epitopes different from this Fv are carried by the single-chain antibody ScFv1 and the single-chain antibody ScFv2, respectively. That is, the multispecific antibody 33 is designed to have an increased binding titer to one of target epitopes while being a trispecific antibody. In more specific examples of these multispecific antibodies 31, 32, and 33, two Fvs in both arms of the antibody can be configured to be specific to a cancer cell, and the single-chain antibody ScFv1 and/or the single-chain antibody ScFv2 can be configured to be specific to an immune cell, and such an antibody can be used for the purpose of inhibiting cancer cell-independent immune cell activation and enhancing cytotoxicity against cancer cells.

In the polypeptide a-31 chain of the multispecific antibody 31, either VH or VL of the single-chain antibody ScFv1 may be linked to the variable region Va1. Similarly, in the polypeptide a-32 chain of the multispecific antibody 32, either VH or VL of the single-chain antibody ScFv2 may be linked to the constant region Ca2. Similarly, in a polypeptide a-43 chain of the multispecific antibody 43, either VH or VL of the single-chain antibody ScFv1 may be linked to the variable region Va1, or either VH or VL of the independent single-chain antibody ScFv2 may be linked to the constant region Ca2.

The sequence and length of a peptide linker linking VH and VL constituting the single-chain antibodies ScFv1 and ScFv2; the form of linking the single-chain antibodies ScFv1 and ScFv2 to the variable region Va1 or the constant region Ca2; and the sequence and length of a peptide linker linking the single-chain antibodies ScFv1 and ScFv2 to the variable region Va1 or the constant region Ca2 are as described in the above “1-6. Multispecific antibodies 21, 22, and 23”.

The specific sequences of the variable region Va1, the constant region Ca1, the variable region Va2, and the constant region Ca2 in the polypeptide a-31 chain, the polypeptide a-32 chain, and the polypeptide a-33 chain and the variable region Vb and the constant region Cb of the polypeptide b chain constituting each of the multispecific antibodies 31 to 33 may each independently be a sequence in either the heavy chain or the light chain.

Therefore, in examples of the multispecific antibodies 31 to 33, similarly to the multispecific antibody 12 of FIG. 5, all the variable region Va1, the constant region Ca1, the variable region Va2, and the constant region Ca2 in the polypeptide a-31 chain, the polypeptide a-32 chain, and the polypeptide a-33 chain may have a heavy-chain sequence, and both the variable region Vb and the constant region Cb of the polypeptide b chain may have a light-chain sequence. In other examples of the multispecific antibodies 31 to 33, similarly to the multispecific antibody 13 of FIG. 7, the variable region Va1 and the variable region Va2 in the polypeptide a-31 chain, the polypeptide a-32 chain, and the polypeptide a-33 chain may have a heavy-chain sequence, the constant region Ca1 and the constant region Ca2 may have a light-chain sequence, and in the polypeptide b chain, the variable region Vb may have a light-chain sequence and the constant region Cb may have a heavy-chain sequence.

As further modified examples of the multispecific antibodies 31 to 33, the peptide linker LL may be replaced with the peptide linker LL-11 including a protease recognition sequence, or the Fc region may be absent.

1-8. Multispecific Antibody 11′ The multispecific antibody of the present invention may be a multispecific antibody in which the peptide linker LL contributing to the formation of its characteristic format is cleaved. The cleavage mode of the peptide linker LL is not particularly limited, and preferred examples thereof include a mode in which a protease sequence introduced into the peptide linker LL is specifically cleaved by a corresponding protease.

Examples of the multispecific antibody of the present invention in such a cleavage mode include a multispecific antibody having a Fab region including one predetermined polypeptide a′ chain, one predetermined polypeptide a″ chain, and two predetermined polypeptide b chains. The predetermined polypeptide a′ chain includes a polypeptide in which a variable region Va1, a constant region Ca1, and a cleavage fragment Lr1′ of a protease recognition sequence Lr1 are linked in the stated order. The predetermined polypeptide a″ chain includes a polypeptide in which a cleavage fragment Lr2′ of a protease recognition sequence Lr2, a variable region Va2, and a constant region Ca2 are linked in the stated order. The predetermined polypeptide b chain includes a polypeptide in which a variable region Vb is linked to a constant region Cb binding to the constant region Ca1 or the constant region Ca2.

As a specific example of such a multispecific antibody of the present invention, FIG. 14 schematically shows an example of an IgD, E, G-type bispecific antibody (multispecific antibody 11′) including a cleavage mode of a peptide linker. The multispecific antibody 11′ has a structure in which the protease recognition sequences Lr1 and Lr2 introduced into the peptide linker LL are specifically cleaved by the corresponding proteases in the multispecific antibody 11 of FIG. 3, whereby the polypeptide a chain is divided into a polypeptide a′ chain and a polypeptide a″ chain, and a cleavage fragment Lr1′ derived from the protease recognition sequence Lr1 and a cleavage fragment Lr2′ derived from the protease recognition sequence Lr2 remain.

Similarly to the content described in the multispecific antibodies 21 to 23 of FIGS. 9 to 11, the specific sequences of the variable region Va1, the constant region Ca1, the variable region Va2, and the constant region Ca2 of the polypeptide a′ chain and the polypeptide a″ chain and the variable region Vb and the constant region Cb of the polypeptide b chain constituting the multispecific antibody 11′ may each independently be a sequence in either the heavy chain or the light chain.

As a further modified example of the multispecific antibody 11′, the Fc region may be absent.

2. DNA

A DNA of the present invention is a DNA encoding a polypeptide a chain including a polypeptide in which a variable region Va1, a constant region Ca1, a peptide linker LL, a variable region Va2, and a constant region Ca2 are linked in the stated order. The DNA of the present invention is used to prepare the above “1. Multispecific antibody”. Therefore, the DNA of the present invention is used together with a DNA encoding a polypeptide b chain including a polypeptide in which a variable region Vb is linked to a constant region Cb binding to the constant region Ca1 or the constant region Ca2.

The polypeptide a chain and the polypeptide b chain are as described in detail in the above “1. Multispecific antibody”. The polypeptide a-11 chain (an embodiment including a protease recognition sequence), the polypeptide a-12 chain (an embodiment in which the heavy-chain/light-chain origin of each region is specified), the polypeptide a-13 chain (an embodiment in which the heavy-chain/light-chain origin of each region is specified), the polypeptide a-21 chain (an embodiment including a single-chain antibody), the polypeptide a-22 chain (an embodiment including a single-chain antibody), and the polypeptide a-23 chain (an embodiment including a single-chain antibody), which are specific embodiments of the polypeptide a chain; and the polypeptide b-12 chain (an embodiment in which the heavy-chain/light-chain origin of each region is specified) and the polypeptide b-13 chain (an embodiment in which the heavy-chain/light-chain origin of each region is specified), which are specific embodiments of the polypeptide b chain, are also as described in detail in the above “1. Multispecific antibody”.

The base sequences of the DNA encoding the polypeptide a chain and the DNA encoding the polypeptide b chain can be appropriately designed by those skilled in the art based on the designs of the polypeptide a chain and the polypeptide b chain. The DNA encoding the polypeptide a chain and the DNA encoding the polypeptide b chain can be obtained by artificial synthesis by genetic engineering.

These DNAs are preferably those in which the codon usage frequency is optimized for the host. For example, when a human cell is used as a host, a DNA in which the codon usage frequency is optimized for the human cell is suitable.

3. Recombinant Vector

Each of the DNA encoding the polypeptide a chain and the DNA encoding the polypeptide b chain described in the above “2. DNA” can be incorporated into an expression vector. An expression vector into which the DNA encoding the polypeptide a chain is incorporated is described as a recombinant vector va, and an expression vector into which the DNA encoding the polypeptide b chain is incorporated is described as a recombinant vector vb.

The schematic diagrams of the recombinant vector va and the recombinant vector vb are shown in FIG. 15. The recombinant vector va and the recombinant vector vb shown in FIG. 15 are examples of those preparing the multispecific antibody 1 of FIG. 1. In FIG. 15, “S” represents a signal sequence, “Va1” represents a DNA encoding the variable region Va1, “Ca1” represents a DNA encoding the constant region Ca1, “LL” represents a DNA encoding the peptide linker LL, “Va2” represents a DNA encoding the variable region Va2, “Ca2” represents a DNA encoding the constant region Ca2, “Vb” represents a DNA encoding the variable region Vb, “Cb” represents a DNA encoding the constant region Cb, “H” represents a DNA encoding a hinge region, “CH2” represents a DNA encoding the constant region CH2, and “CH3” represents a DNA encoding the constant region CH3.

The recombinant vector va includes a control factor such as a promoter operably linked to the DNA encoding the polypeptide a chain. Similarly, the recombinant vector vb includes a control factor such as a promoter operably linked to the DNADNA encoding the polypeptide b chain. Typical examples of the control factor include a promoter, but the control factor may further include transcription elements such as an enhancer, a CCAAT box, a TATA box, and an SPI site, as necessary. The expression “operably linked” means that various control factors such as a promoter and an enhancer that control the DNA encoding the polypeptide a chain or the polypeptide b chain are linked to the DNA of the present invention in a state of being capable of operating in a host cell.

As the expression vector, those constructed for recombination from phage, plasmid, or virus capable of autonomously growing in the host are suitable. Such expression vectors are known, and examples thereof include pUC vectors, pBluescript vectors, pET vectors, pGEX vectors, pEX vectors, and pCAGGS vectors. The expression vector may be used by selecting an appropriate combination with a host cell.

4. Transformant

A transformant of the present invention is obtained by transforming a host by using the DNA encoding the polypeptide a chain and the DNA encoding the polypeptide b chain described in the above “2. DNA” or the recombinant vector va and the recombinant vector vb described in the above “3. Recombinant vector”.

As a host used to produce a transformant, a host can be used without particular limitation from prokaryotic cells and eukaryotic cells as long as it can introduce a gene, autonomously proliferate, and express the multispecific antibody of the present invention. Specific examples of the host cell include mammalian cells such as CHO cells, N50 cells, SP2/0 cells, Expi293 cells, HEK293 cells, COS cells, and PER.C6 cells; fungi such as yeast; and bacteria such as Escherichia coli (E. coli).

The transformant of the present invention can be produced by introducing the DNA or recombinant vector into a host. The method for introducing these nucleic acid species is not particularly limited as long as a target gene can be introduced into the host. The place where the DNA is introduced is also not particularly limited as long as a target gene can be expressed, and may be on a plasmid or on a genome. Specific examples of the method for introducing the DNA or recombinant vector include a recombinant vector method and a genome editing method.

Conditions for introducing the DNA or the recombinant vector into the host may be appropriately set according to the introduction method, the type of the host, and the like. When the host is an animal cell, for example, a polyethyleneimine method, an electroporation method, a calcium phosphate method, a lipofection method, and the like are mentioned. When the host is a fungus, for example, an electroporation method, a spheroplast method, a lithium acetate method, and the like are mentioned. When the host is a bacterium, for example, a method using a competent cell by a calcium ion treatment, an electroporation method, and the like are mentioned.

As the ratio of the DNA or recombinant vector to be introduced into the host, the ratio of the DNA encoding the polypeptide a chain or the recombinant vector va with respect to 1 mol of the DNA encoding the polypeptide b chain or the recombinant vector vb is, for example, 0.4 mol or more or 0.5 mol or more, preferably 0.7 mol or more, more preferably 0.9 mol or more or 1 mol or more, and further preferably 1.2 mol or more, 1.25 mol or more, 1.35 mol or more, 1.4 mol or more, or 1.5 mol or more. The upper limit of the range of the molar ratio of the recombinant vector va with respect to 1 mol of the recombinant vector vb is not particularly limited, and is, for example, 3 mol or less, 2.5 mol or less, 2 mol or less, or 1.8 mol or less. The use in such amounts is preferable from the viewpoint of reducing the generation amount of the tetramer 12BQ when the tetramer 12BQ of the polypeptide b-12 chain shown in FIG. 6 is generated as a by-product.

5. Method for Producing Multispecific Antibody

A method for producing a multispecific antibody of the present invention includes an antibody production step of culturing the transformant of the present invention.

The culture conditions in the antibody production step may be appropriately set in consideration of the nutritional physiological properties of the host, and liquid culture is preferably mentioned. In view of industrial production, the culture is preferably performed under ventilation and stirring conditions.

In the antibody production step, the polypeptide a chain and the polypeptide b chain are expressed and spontaneously associated with each other to constitute the multispecific antibody of the present invention. For example, in the antibody production step in the method for producing the multispecific antibody 1 shown in FIG. 1, the polypeptide a chain and the polypeptide b chain shown in FIG. 2 are expressed and spontaneously associated with each other to constitute the multispecific antibody 1. Depending on the specific sequences of the polypeptide a chain and the polypeptide b chain, a by-product may be generated as shown in FIG. 6, but the by-product does not exhibit immune activity.

When a multispecific antibody produced in the antibody production step includes a protease recognition sequence in the peptide linker LL, the multispecific antibody can be subjected to a linker cleavage step of cleaving the peptide linker LL using a protease corresponding to the protease.

For example, when the peptide linker LL in the multispecific antibody produced in the antibody production step includes the protease recognition sequence Lr1 on the constant region Ca1 side and the protease recognition sequence Lr2 on the variable region Va2 side as shown in FIG. 3, in the linker cleavage step, the peptide linker LL is cleaved using proteases corresponding to the protease recognition sequence Lr1 and the protease recognition sequence Lr2, respectively.

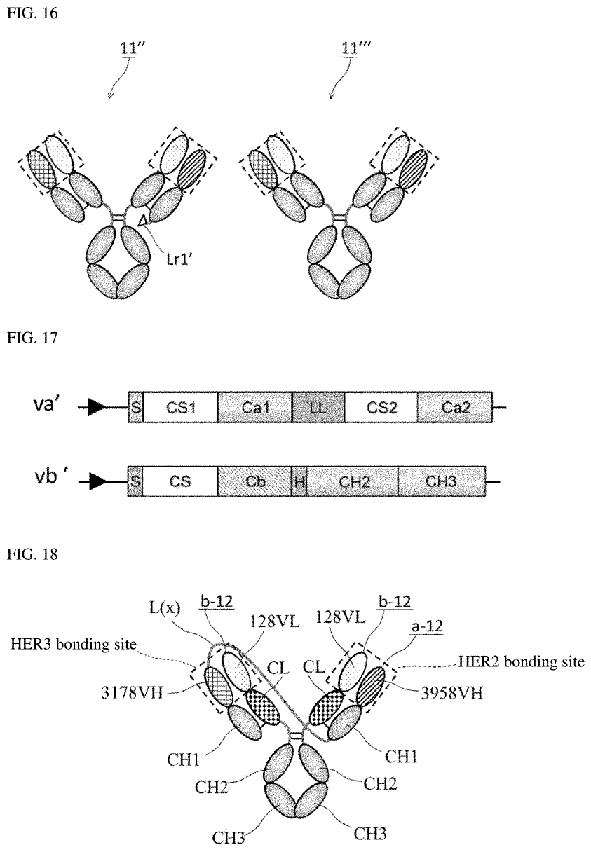

In the case of using, as the protease recognition sequence Lr1 and protease recognition sequence Lr2, those which are designed so that the cleavage fragment Lr1′ and the cleavage fragment Lr2′ remain on the multispecific antibody side by cleavage using the corresponding proteases, respectively, the multispecific antibody 11′ shown in FIG. 14 is obtained by cleavage of the peptide linker LL. In the case of using, as the protease recognition sequence Lr1 and protease recognition sequence Lr2, those which are designed so that the cleavage fragment Lr1′ and/or the cleavage fragment Lr2′ do not remain on the multispecific antibody side by cleavage using the corresponding proteases, respectively, a multispecific antibody 11″ (an embodiment may be employed in which Lr1′ does not exist and Lr2′ remains although not illustrated) or a multispecific antibody 11′″ shown in FIG. 16 is obtained by cleavage of the peptide linker LL.

The multispecific antibody of the present invention obtained by the antibody production step or the linker cleavage step can be produced by further performing a purification step. As a method used in the purification step, a known antibody purification method can be used, and examples thereof include centrifugation, affinity chromatography (such as protein A affinity chromatography or protein G affinity chromatography), size exclusion chromatography, ion exchange chromatography (cation exchange chromatography or anion exchange chromatography), hydrophobic interaction chromatography, gel electrophoresis, and dialysis, and preferably, protein A affinity chromatography and ion exchange chromatography (preferably cation exchange chromatography) can be used in combination.

Since the multispecific antibody of the present invention is designed not to be accompanied by a by-product exhibiting immune activity, a special and complicated separation step used in usual purification of the multispecific antibody is not required in the purification step. That is, the method for producing a multispecific antibody of the present invention can be simplified, and is extremely advantageous in industrial production.

6. Multispecific Antibody Preparation Kit

The present invention also provides a multispecific antibody preparation kit for preparing the above “1. Multispecific antibody”. The multispecific antibody preparation kit of the present invention can be used for preparing the above “3. Recombinant vector” or the above “4. Transformant” or for carrying out the above “5. Method for producing multispecific antibody”.

The multispecific antibody preparation kit of the present invention includes: an expression vector va′ including a cloning site CS1 for incorporating a variable region Va1, a DNA encoding a constant region Ca1, a DNA encoding a peptide linker LL, a cloning site CS2 for incorporating a variable region Va2, and a DNA encoding a constant region Ca2 in the stated order; and an expression vector vb′ including a cloning site CS for incorporating a variable region Vb, and a DNA encoding a constant region Cb binding to the constant region Ca1 or the constant region Ca2.

FIG. 17 schematically shows expression vectors included in a multispecific antibody preparation kit of the present invention. In the expression vector va′, the restriction enzyme sites included in the cloning site CS1 and the cloning site CS2 are designed to be specific to mutually different restriction sequences so that the variable region Va1 and the variable region Va2 can be incorporated, respectively. Each of the cloning sites CS1, CS2, and CS may include one restriction enzyme site or may be a multiple cloning site including two or more restriction enzyme sites. As restriction enzyme sites included in these cloning sites, known restriction enzyme sites are appropriately selected. As the multiple cloning site, a multiple cloning site or the like included in a known cloning vector or expression vector may be used as it is, or a multiple cloning site obtained by appropriately modifying a known multiple cloning site may be used.

The method for incorporating the variable regions Va1, Va2, and Vb into the cloning sites CS1, CS2, and CS can be appropriately determined by those skilled in the art based on a known cloning method using restriction enzymes corresponding to the cloning sites CS1, CS2, and CS.

7. Diagnostic Composition and Pharmaceutical Composition

The multispecific antibody of the present invention can be used for any application that utilizes the ability to specifically bind to two or more different epitopes. Examples of an embodiment in which the specific binding ability of the multispecific antibody of the present invention is utilized include an embodiment in which a plurality of antigens (cytokine and tumor) are targeted, an embodiment in which different epitopes possessed by the same tumor or the same viral antigen are targeted, and an embodiment in which two target cells are crosslinked (for example, an embodiment in which immune effector cells are brought into close contact with a particular tumor associated antigen to promote cell killing). Therefore, the multispecific antibody of the present invention is useful for diagnostic applications and pharmaceutical (so-called antibody drug) applications.

Therefore, the multispecific antibody of the present invention can be used as an active ingredient of a diagnostic agent (sensing component) or a pharmaceutical composition. That is, the present invention provides a diagnostic agent including the multispecific antibody described in the above “1. Multispecific antibody” and a pharmaceutical composition including the multispecific antibody described in the above “1. Multispecific antibody”.

Examples of the diagnostic agent of the present invention include those in which a multispecific antibody constitutes a composition together with other components (for example, a buffering agent, a suspending agent, a stabilizer, a preservative, an antiseptic agent, and the like) used in a general diagnostic agent composition, and those in which a multispecific antibody is immobilized on the surface of an insoluble carrier (a particle or a substrate).

Examples of the pharmaceutical composition of the present invention include those in which a multispecific antibody constitutes a composition together with other components (for example, an excipient, a buffering agent, a suspending agent, a stabilizer, a preservative, an antiseptic agent, physiological saline, and the like) used in a general pharmaceutical composition.

### EXAMPLES

Hereinafter, the present invention will be specifically described by means of Examples; however, the present invention is not to be construed as being limited to the following Examples. Hereinafter, the multispecific antibody of the present invention, that is, the monoclonal antibody of the multispecific antibody having a Fab region including one polypeptide a chain (including the polypeptide in which the variable region Va1, the constant region Ca1, the peptide linker LL, the variable region Va2, and the constant region Ca2 are linked in the stated order) and two polypeptide b chains (including the polypeptide in which the variable region Vb is linked to the constant region Cb binding to the constant region Ca1 or the constant region Ca2) is also referred to as Trimeric Bi-specific Monoclonal Antibody Common Light Chain (TribsMab CLC).

### Test Example 1: Design and Production of Multispecific Antibody (Anti-HER2×HER3 Bispecific Antibody-1)

(1) Design of Anti-HER2×HER3 Bispecific Antibody (HER2×HER3 TribsMab CLC)

A bispecific antibody corresponding to the multispecific antibody 12 of FIG. 5 was designed. In this test example, the bispecific antibody was designed to target HER2 and HER3 expressed on the surface of tumor cells. As for the variable regions (portions surrounded by dashed lines in FIG. 5), the same sequences as those of the variable regions of MCLA-128 (Cancer Cell 33, 922-936 (2018)) known as the anti-HER2×Her3 bispecific antibody, that is, sequences of the heavy-chain variable region 3958VH (specific to HER2) and the heavy-chain variable region 3178VH (specific to HER3) of MCLA-128, and the light-chain variable region 128VL of MCLA-128 were adopted. As for the constant region, a sequence derived from the human IgG1 class was adopted. As a sequence corresponding to the peptide linker LL of FIG. 5, peptide linkers with different lengths each having GGGGS as a basic sequence and including or not including the HRV3C protease recognition sequence (LEVLFQGP) were designed.

The correspondence relationship between the domains of the multispecific antibody 12 of FIG. 5 and the bispecific antibody designed in the present test example is shown in the following Tables 1 and 2, and the schematic diagram of the bispecific antibody designed in the present test example is shown in FIG. 18. Approximate calculation of specific lengths (A) and specific sequences of the peptide linkers with different lengths are as shown in Table 1. Each peptide linker is represented by L(x), “x” in parentheses indicates the total number of amino acid residues constituting the peptide linker in the case of those containing a protease recognition sequence (for example, a peptide linker which includes a protease recognition sequence and in which the total number of amino acid residues constituting the peptide linker is 68, is indicated as “L(68)”), and “delP” is added in the case of those not containing a protease recognition sequence.

TABLE 1

| | a-12 chain | | | | |
|---|---|---|---|---|---|
| | VHa1 | CHa1 | LL | VHa2 | CHa2 |
| Example 1 | MCLA-128 | Human | L (68); ≈240 Å | MCLA-128 | Human |
| | 3958VH | IgG1 | (G4S)8LEVLFQGP(G4S)4 | 3178VH | IgG1 |
| Example 2 | | CH1 | L (53); ≈190 Å | | CH1 |
| | | | (G4S)6LEVLFQGP(G4S)3 | | |
| Example 3 | | | L (38); ≈130 Å | | |
| | | | (G4S)4LEVLFQGP(G4S)2 | | |
| Example 4 | | | L (23); ≈81 Å | | |
| | | | (G4S)2LEVLFQGP(G4S) | | |
| Example 5 | | | L (68, delP); ≈240 Å | | |
| | | | (G4S)13(G2S) | | |

TABLE 2

| | b-12 chain | | | |
|---|---|---|---|---|
| | VLb | CLb | CH2 | CH3 |
| Examples | MCLA-128 | Human | Human | Human |
| 1 to 5 | 128VL | IgG1 CL | IgG1 CH2 | IgG1 CH3 |

(2) Construction of Recombinant Vector for Bispecific Antibody Expression

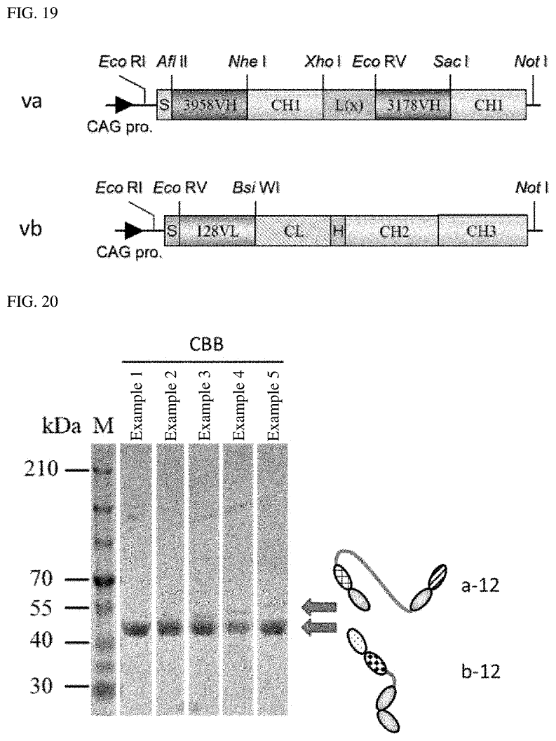

The schematic diagrams of a recombinant vector va for a-12 chain and a recombinant vector vb for b-12 chain for expressing the bispecific antibodies designed in the above (1) are shown in FIG. 19. In the drawing, “S” represents a signal sequence, and “H” represents a hinge site. As an expression vector incorporating a DNA encoding each chain, pCAGGS, which is an expression vector for mammalian cells having a promoter of a chicken-derived β-actin gene and a CMV-IE enhancer (CAG promoter) derived from cytomegalovirus, was used. A leader sequence for secretory expression of an antibody was incorporated on the N-terminal side of the DNA encoding each chain. The sequences of the obtained recombinant vectors va and vb were confirmed by sequencing the base sequences.

(3) Preparation of Transformant

Human embryonic kidney cell Expi293F cells were used as a host, and transfection was performed by a polyethyleneimine method. All the cultures were performed under the conditions of 37° C., 5% CO2, and 125 rpm.

A solution obtained by adding 6 μg of the expression vector va and 12 μg of the expression vector vb (the expression vector introduction ratio va:vb is about 0.5:1 on a molar basis) to 900 μL of Opti-MEM® (Thermo) and a solution obtained by adding 75 μL of PEI-MAX (Polysciences) to Opti-MEM® were prepared. These two solutions were mixed and left to stand still for 20 minutes, and then added to 16.2 mL of HE400 (Gmep) to prepare a transfection solution. Host cells were cultured in an Erlenmeyer flask containing 30 mL of HE200 medium (Gmep), and when the number of cells reached 3-5×10⁶ cells/mL and the survival rate was 95% or more, the cell culture solution was dispensed into a 50 mL sample tube so that the number of cells was 45×10⁶ cells. After the sample tube was centrifuged at 1,500 rpm for 5 minutes, the culture solution was removed, and the cells were suspended in the transfection solution. Each 3 mL of the cell suspension was seeded on a 6-well plate and cultured for 20 hours.

(4) Production of Bispecific Antibody

To each well, 7.5 μL of a 0.5 M sodium valproate aqueous solution and 12 μL of a 1 M sodium propionate aqueous solution were added, and the mixture was further cultured for 6 days. The culture conditions are as described in the above (3). The supernatant after culture was subjected to Western blotting using polyacrylamide gel electrophoresis (SDS-PAGE) and an anti-human Fc antibody to confirm expression of the bispecific antibody.

(5) Purification of Bispecific Antibody

(5-1) Protein A Affinity Purification

The culture solution was subjected to a centrifugation operation at 4° C. and at 6,000 rpm for 10 minutes to remove cells, and the culture supernatant was filtered through a filter (Millex-HP, Millipore) having a pore size of 0.45 μm. The culture supernatant filtrate was added to KANEKA Kan CapA™ (column volume: 500 μL) pre-equilibrated with TBS (25 mM Tris, 150 mM NaCl, pH 7.2), followed by washing with TBS and elution with Gentle Ag/Ab Elution Buffer (Thermo).

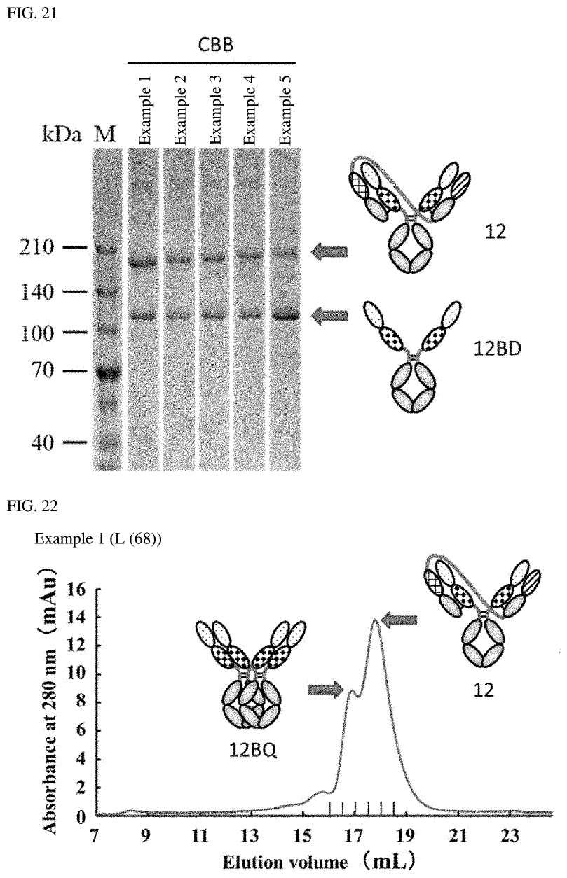

Each eluted fraction was developed by SDS-PAGE under reduction conditions and under non-reduction conditions and subjected to CBB staining. Results under reduction conditions are shown in FIG. 20, and results under non-reduction conditions are shown in FIG. 21.

The molecular weight was calculated from the mobility of the band recognized in FIG. 20 (under reduction conditions), and the molecular weights based on the amino acid sequences of the polypeptide a-12 chain and the polypeptide b-12 chain were compared. Results are shown in Table 3. From the results of Table 3, the polypeptide a-12 chain and the polypeptide b-12 chain constituting the target bispecific antibody (multispecific antibody 12) could be confirmed.

TABLE 3

| | | | Molecular | |
|---|---|---|---|---|
| | | | weight based | Molecular |
| | | | on amino acid | weight based |
| | | | sequence | on mobility |
| | | | (Da) | (Da) |
| Example 1 | a-12 chain | L (68) | 56,019 | 56,900 |
| Example 2 | a-12 chain | L (53) | 55,073 | 55,600 |
| Example 3 | a-12 chain | L (38) | 54,127 | 54,300 |
| Example 4 | a-12 chain | L (23) | 53,181 | 53,100 |
| Example 5 | a-12 chain | L (68, delP) | 55,651 | 59,600 |
| Examples | b-12 chain | | 51,678 | 51,800 |
| 1 to 5 | | | | |

As a result of deriving the molecular weight based on FIG. 21 (under non-reduction conditions), it was found that a dimer 12BD of the polypeptide b-12 chain exists as a main by-product in addition to the target bispecific antibody (multispecific antibody 12).

(5-2) Analysis of Product by Gel Filtration Chromatography

Fractions containing the target bispecific antibody (multispecific antibody 12) obtained in the above 5-1 were added to a gel filtration chromatography column Superdex 200 10/300 GL (GE Healthcare) equilibrated with PBS. Elution was performed using AKTA Prime plus (GE Healthcare) under the condition of a flow rate of 0.5 mL/min. As a molecular weight marker, Ferritin, Conalbumin, Aldolase, and Ovalbumin were used.

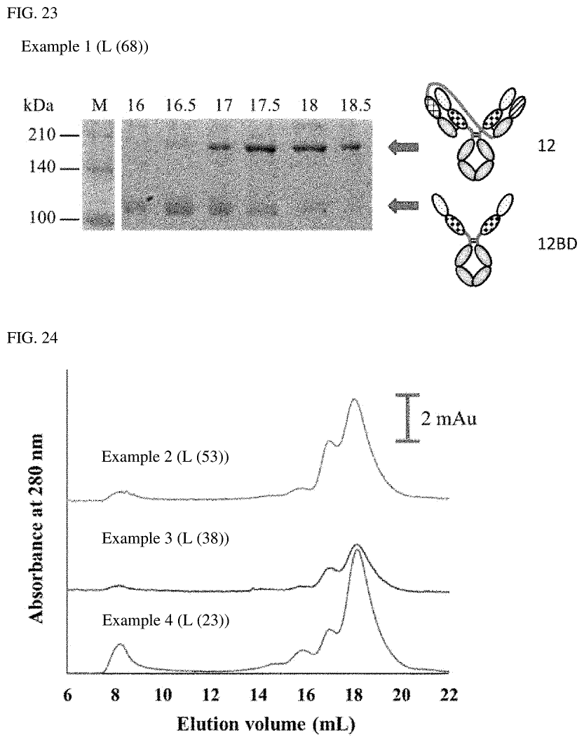

Results of gel filtration chromatography for Example 1 are shown in FIG. 22. Results of SDS-PAGE development (under non-reduction conditions) of 16 mL to 18.5 mL of the eluted fraction in the same manner as in the above 5-1 are shown in FIG. 23.

In the gel filtration chromatogram of FIG. 22, a peak of the bispecific antibody (multispecific antibody 12) and a peak seen as a tetramer 12BQ of the polypeptide b-12 chain were recognized. This inference is based on the fact that the peak of the multispecific antibody 12 in FIG. 22 corresponds to the band in which the multispecific antibody 12 is recognized in FIG. 23, and the peak seen as a tetramer 12BQ in FIG. 22 corresponds to the band in which the dimer BD (detected in a state where the tetramer 12BQ is dissociated according to the conditions during SDS-PAGE) is recognized in FIG. 23. That is, it was found that, together with the target multispecific antibody 12, one seen as a tetramer 12BQ was generated as a by-product.

Each peak area in FIG. 22 was calculated using PrimeView Evaluation (GE Healthcare). As a result, the peak area ratio of the multispecific antibody 12 was about 67%, and the peak area ratio of the tetramer 12BQ was about 32%.

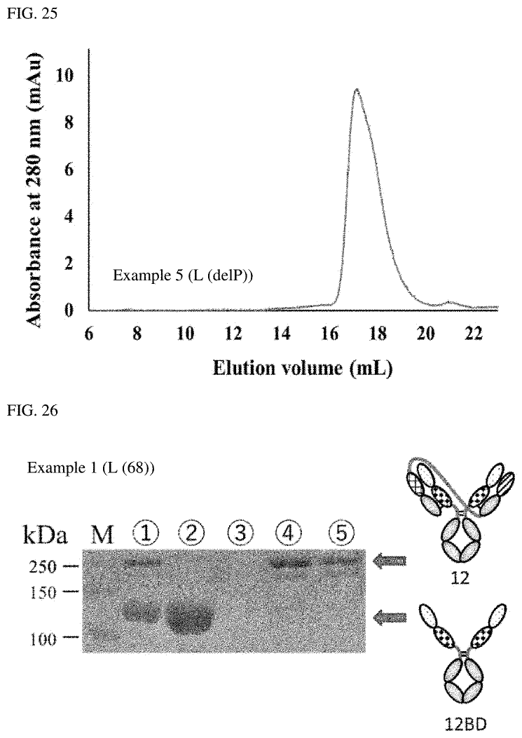

Results of subjecting the multispecific antibodies 12 of Examples 2 to 4 to gel filtration chromatography in the same manner are shown in FIG. 24, and results of subjecting the multispecific antibody 12 of Example 5 in the same manner are shown in FIG. 25.

As shown in FIGS. 22 and 24, in the preparation of the multispecific antibody 12 of Example 1 in which the peptide linker having a protease recognition sequence is the longest, the production ratio of the by-product was the smallest, and it was recognized that the production ratio of the by-product tended to increase as the length of the peptide linker having a protease recognition sequence was shortened (Examples 2 to 4). That is, it was found that when a peptide linker having a protease recognition sequence is used, the production amount of by-products tends to decrease as the length of the peptide linker increases.

On the other hand, as shown in FIG. 25, when the peptide linker did not have a protease recognition sequence (Example 5), production of a by-product was not recognized.

(5-3) Affinity Purification Using IgG-CH1 Binding Carrier

Fractions containing the target bispecific antibody (multispecific antibody 12) obtained in the above 5-1 were added to CaptureSelect™ IgG-CH1 Affinity Matrix (Thermo) (column volume: 250 μL) equilibrated with PBS, washed with PBS, and then eluted using an acetate buffer (20 mM CH₃COOH, 150 mM NaCl, pH 3.5). Each eluted fraction in the purification process was developed by SDS-PAGE (under non-reduction conditions) in the same manner as in the above 5-1. Results are shown in FIG. 26. In FIG. 26, Lane 1 shows after the protein A affinity purification and before IgG-CH1 binding carrier generation, Lane 2 shows the flow-through fraction, Lane 3 shows the washed fraction, Lane 4 shows the eluted fraction 1, Lane 5 shows the eluted fraction 2.

As clearly shown from FIG. 26, since the tetramer BQ as a by-product was eluted into the flow-through fraction, these by-products could be separated from the target multispecific antibody 12.

(5-4) Affinity Purification Using Cation Exchange Chromatography

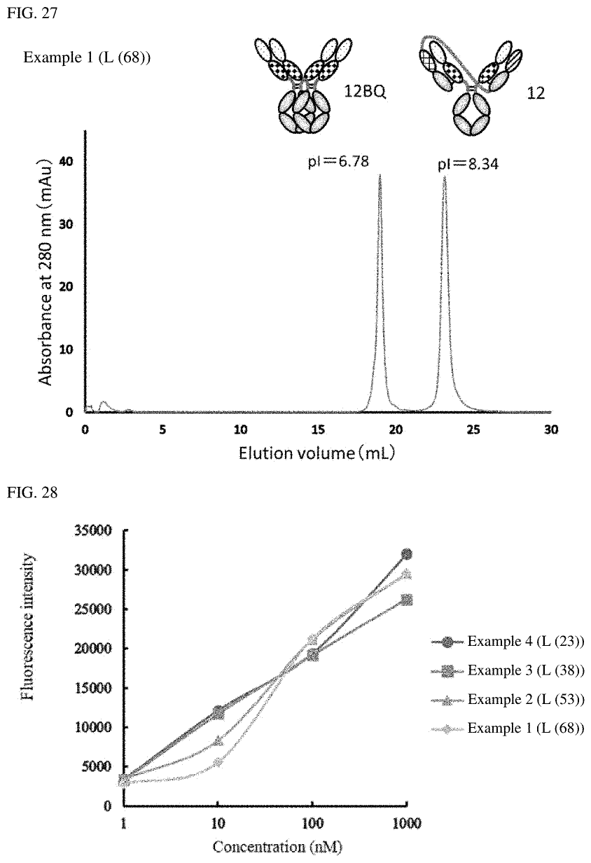

Fractions containing the target bispecific antibody (multispecific antibody 12) obtained in the above 5-1 were added to a cation exchange column Resource S (GE Healthcare) using AKTA Prime plus, and elution was performed using a 10 mM IVIES buffer solution (pH 6.0) as a running buffer and a 10 mM IVIES buffer solution (pH 6.0) containing 1 M NaCl as an elution buffer under gradient conditions in which the concentration of the elution buffer was finally 40%. Results are shown in FIG. 27.

As clearly shown from FIG. 27, the tetramer BQ as a by-product could be clearly separated from the target multispecific antibody 12.

### Test Example 2: Activity Evaluation of Multispecific Antibody (Anti-HER2×HER3 Bispecific Antibody-I)

(1) Binding Activity Evaluation-1

For the multispecific antibodies 12 of Examples 1 to 5 prepared in Test Example 1, binding activity evaluation by flow cytometry to HER2 and HER3-positive human mammary adenocarcinoma cells MCF-7 was performed as follows.

MCF-7 was released from the dish by using trypsin/EDTA (0.25 w/v % trypsin-1 mmol/l EDTA.4Na solution, containing phenol red, Gibco), and cells were collected by centrifugation at room temperature and at 1,000 rpm for 5 minutes. The cells were dispensed into a sample tube by 5×10⁵ cells. After washing once with 0.1% NaN₃/PBS, each of the multispecific antibody 12, the 3958 antibody (anti-HER2 antibody), and the 3178 antibody (anti-HER3 antibody) (500 nM) as a primary antibody was added and reacted in ice for 30 minutes. After washing once, 1 μL of a 2 mg/mL anti-human IgG (Fc-specific)-FITC antibody (Sigma Aldrich) as a secondary antibody and 499 μL of 0.1% NaN₃/PBS were added and reacted in ice for 30 minutes. After washing once, the cells were suspended in 500 μL of 0.1% NaN₃/PBS, filtered through a nylon filter, and dispensed into a plastic tube. Flow cytometric analysis was performed using BD Accuri™ C6 (BD Biosciences). As a result, it was confirmed that all the antibodies of Examples 1 to 5 showed the binding activity with respect to HER2 and HER3-positive human mammary adenocarcinoma cells MCF-7.

(2) Binding Activity Evaluation-2

For the multispecific antibodies 12 of Examples 1 to 4 having different peptide linker lengths, the binding activity to MCF-7 was evaluated by flow cytometry in the same manner as in the above (1), except that the concentration of each of these antibodies was changed to 1 nM, 10 nM, 100 nM, or 1000 nM instead of 500 nM. Results are shown in FIG. 28.

As shown in FIG. 28, a difference in binding activity was recognized when the antibody concentration was 10 nM. Specifically, the fluorescence intensities of Example 4 (L(23)) and Example 3 (L(38)) were almost the same, but the fluorescence intensities thereof were 1.5 times the fluorescence intensity of Example 2 (L(53)) and 2 times the fluorescence intensity of Example 1 (L(68)).

The reason why the difference in binding activity due to the difference in length of the peptide linker is observed is considered to be due to a difference in binding mode. HER2 and HER3 present on the surface of MCF-7 may be present alone or may be present as a heterodimer, and it is considered that the proportion of HER2 and HER3 present as a heterodimer is particularly high on a cell surface where expression levels of HER2 and HER3 are considered to be equal as in the case of MCF-7. It is considered that, since the movable range of the Fab arm of the multispecific antibody is limited as the length of the peptide linker is shorter, the multispecific antibody easily bivalently binds to the heterodimer of HER2 and HER3, and thus the binding property to cells is enhanced as compared with the case of monovalently binding. It is considered that, under a situation where the antibody concentration is low, the influence of the binding form due to the bivalent binding is greatly exhibited.

(3) Bispecificity Evaluation

For the multispecific antibody 12 of Example 1, bispecificity evaluation using a surface plasmon resonance (SPR) method was performed as follows.

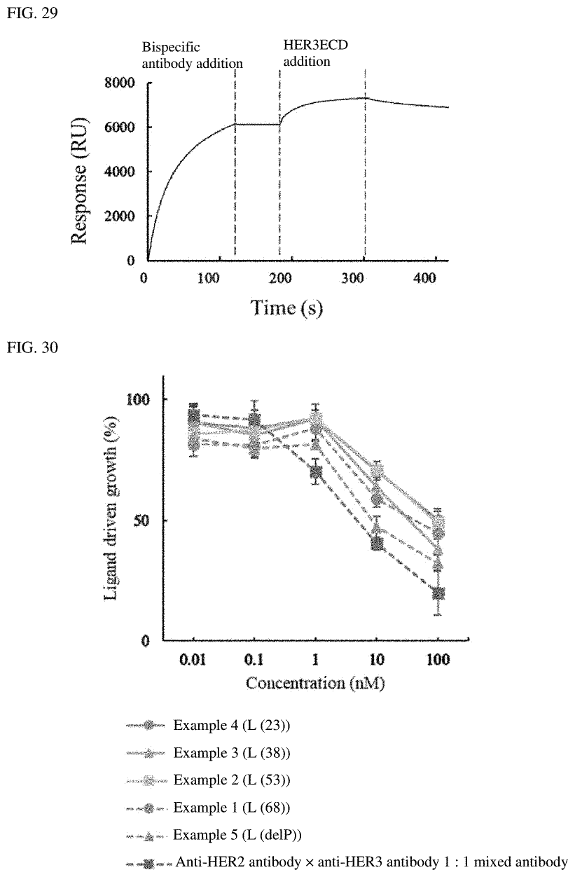

An Fc-fused HER2 extracellular domain (ECD) was immobilized on the surface of a sensor chip CM5 for Biacore using an amine coupling kit (GE Healthcare). In an environment at 25° C., the multispecific antibody 12 (1 μM) of Example 1 and Fc-fused HER3ECD (1 μM) were added to the Fc-fused HER2ECD immobilized sensor chip in this order at a flow rate of 20 μL/min, and SPR analysis was performed using Biacore 3000 (GE Healthcare). As a running buffer solution, PBS (PBST) containing 0.005% Tween-20 was used. Results are shown in FIG. 29.

As shown in FIG. 29, when the bispecific antibody (multispecific antibody 12) of Example 1 was added to the Fc-fused HER2ECD immobilized sensor chip, the response indicating the increase in mass on the sensor chip increased, and when the Fc-fused HER3ECD was added, the response similarly increased. That is, it could be confirmed that the bispecific antibody (multispecific antibody 12) of Example 1 has bispecificity for HER2 and HER3 that are antigens.

(4) Cell Growth Inhibitory Potency

MCF-7 was cultured using D-MEM (Dulbecco's Modified Eagle Medium, Sigma Aldrich) containing 10% FBS. Cells were collected from the dish by using trypsin/EDTA (0.25 w/v % trypsin-1 mmol/l EDTA.4Na solution, containing phenol red, Gibco), suspended in RPMI 1640 (Roswell Park Memorial Institute medium, Sigma Aldrich) containing 1% FBS so as to be 6.25×10³ cells/mL, and seeded into wells of a 96-well flat bottom culture plate in 80 μL each. 10 μL each of recombinant human heregulin β-1 (carrier-free) (TONBO biosciences) diluted to 0.1 ng/μL with PBS was added to each well, and further 10 μL each of the multispecific antibodies 12 of Examples 1 to 5 prepared to have concentrations of 0.1 nM, 1 nM, 10 nM, 100 nM and 1000 nM, the 3958 antibody (anti-HER2 antibody), and the 3178 antibody (anti-HER3 antibody) was added to each well. After 6 days of culture under the conditions of 37° C. and 5% CO2, viable cells were colorimetrically quantified by 344,5-dimethylthiazol-2-yl)-5-(3-carboxymethoxyphenyl)-2-(4-sulfophenyl)-2H-tetrazolium inner salt (MTS) assay using CellTiter 96 AQueous One Solution Reagent (Promega). The absorbance at 490 nm was measured using iMark Microplate Reader (BioRad), the absorbance at 630 nm was subtracted as a background, and the cell proliferation rate with respect to MCF-7 was calculated based on the following formula (A490 target maximum control refers to the absorbance of MCF-7 when no antibody is added).

Results are shown in FIG. 30.

$$Cell proliferation rate ( \% ) = \frac{\begin{matrix}A 490 measurement va1ue - \\ A 490 background\end{matrix}}{\begin{matrix}A 490 target maximum contro1 - \\ A 490 background\end{matrix}} \times 100 \qquad [ Mathematical Formula 1 ]$$

As shown in FIG. 30, it was found that all the bispecific antibodies (multispecific antibodies 12) of Examples 1 to 5 showed cell growth inhibitory potency with respect to MCF-7, and the cell proliferation rate decreased with an increase in the antibody concentration. That is, it was shown that all the bispecific antibodies (multispecific antibodies 12) of Examples 1 to 5 inhibited the proliferation signal of MCF-7. Since no significant difference in cell growth inhibitory potency was recognized among Examples 1 to 5, it is considered that there is no significant difference in growth inhibitory potency due to the difference in linker length.

### Test Example 3: Examination of Production Conditions of Multispecific Antibody

A multispecific antibody was prepared in the same manner as in Example 1, except that the introduction ratio of the recombinant vector va and the recombinant vector vb prepared in Test Example 1 was changed, and the generation amount of the tetramer 12BQ was confirmed in the same manner as in (5-2) of Test Example 1 on a gel filtration chromatogram. The introduction ratio (weight basis) of the recombinant vector va and the recombinant vector vb adopted in the present test example is shown in the following table. The introduction ratio (weight basis) of the recombinant vector va and the recombinant vector vb shown in the following table is substantially the same as the introduction ratio on a molar basis. In the following table, the introduction ratio in Example 1 prepared in Test Example 1 is also described.

TABLE 4

| | Recombinant vector introduction ratio |
|---|---|
| | va:vb |
| Example 1 | 0.5:1 |
| Example 6 | 1:1 |
| Example 7 | 1.25:1 |
| Example 8 | 1.5:1 |

As a result, the generation amount of the tetramer 12BQ decreased in the order of Example 1, Example 6, and Example 7, and was substantially the same in Example 7 and Example 8. That is, it was found that, as the introduction amount of the recombinant vector va is increased with respect to the theoretical introduction amount of the recombinant vector va and the recombinant vector vb (Example 1), the generation amount of the tetramer 12BQ as a by-product tends to decrease.

### Test Example 4: Design and Production of Multispecific Antibody (Anti-HER2×HER3 Bispecific Antibody-2)

(1) Design of HER2×HER3 Bispecific Antibody

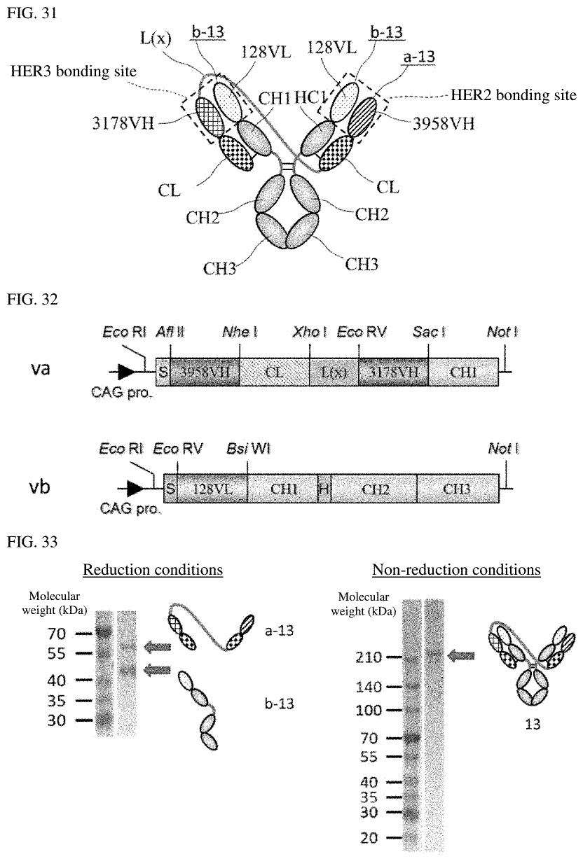

A bispecific antibody corresponding to the multispecific antibody 13 of FIG. 7 was designed. Specifically, the multispecific antibody 13 (Example 9) was designed in the same manner as in Example 1 in Test Example 1, except that the heavy-chain constant region CHa1 and the light-chain constant region CHb of the multispecific antibody 12 prepared in Example 1 of Test Example 1 were interchanged, and the heavy-chain constant region CHa2 and the light-chain constant region CHb were interchanged. The correspondence relationship between the domains of the multispecific antibody 13 (Example 9) of FIG. 7 and the bispecific antibody designed in the present test example is shown in the following Tables 5 and 6, and the schematic diagram of the bispecific antibody (Example 9) designed in the present test example is shown in FIG. 31.

TABLE 5

| | a-13 chain | | | | |
|---|---|---|---|---|---|
| | VHa1 | CLa1 | LL | VHa2 | CLa2 |
| Example 9 | MCLA-128 | Human | L (68): | MCLA-128 | Human |
| | 3958VH | IgG1 CL | (G4S)8LEVLFQGP(G4S)4 | 3178VH | IgG1 CL |

TABLE 6

| | a-13 chain | | | |
|---|---|---|---|---|
| | VLb | CHb | CH2 | CH3 |
| Example 9 | MCLA-128 | Human | Human | Human |
| | 128VL | IgG1 CH1 | IgG1 CH2 | IgG1 CH3 |

(2) Construction of Recombinant Vector for Bispecific Antibody Expression

The schematic diagrams of a recombinant vector va for a-13 chain and a recombinant vector vb for b-13 chain for expressing the bispecific antibodies designed in the above (1) are shown in FIG. 32. These recombinant vectors are also designed in the same manner as the recombinant vectors prepared in Example 1 in Test Example 1, except that the region encoding the heavy-chain constant region CHa1 of the multispecific antibody 12 in the recombinant vector for expressing the bispecific antibody of Example 1 in Test Example 1 and the region encoding the light-chain constant region CHb were interchanged, and the region encoding the heavy-chain constant region CHa2 and the region encoding the light-chain constant region CHb were interchanged.

(3) Preparation, Production, and Purification of Transformant

The preparation, production, and purification (protein A affinity purification) of the transformant were performed in the same manner as in Test Example 1. Results of SDS-PAGE development of the purified fraction under reduction conditions and under non-reduction conditions in the same manner as in Test Example 1 are shown in FIG. 33.

As shown in comparison between FIG. 33 (Example 9) and FIG. 21 (Example 1), according to Example 9, no by-product of the dimer (a dissociation product of the tetramer) was recognized, and almost only the target bispecific antibody (multispecific antibody 13) could be confirmed.

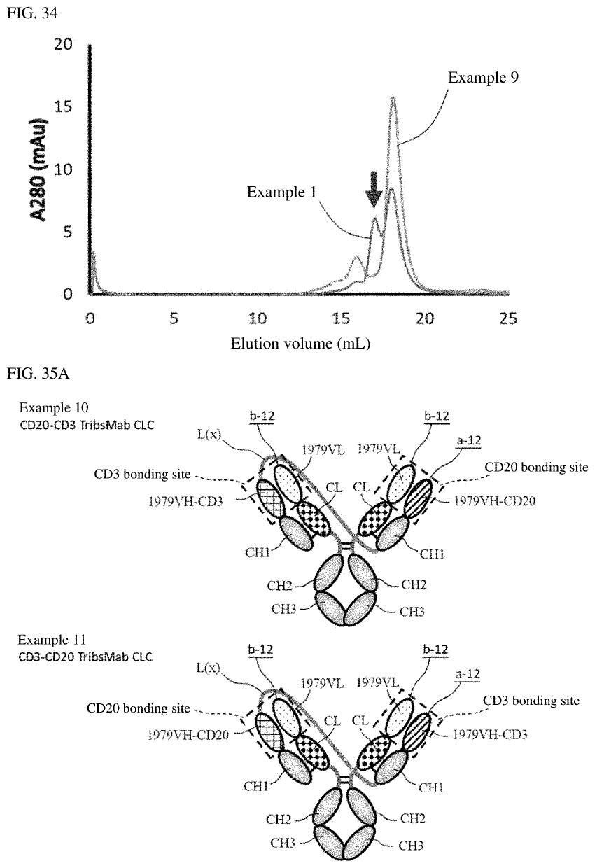

Results of gel filtration chromatography analysis of the purified fraction in the same manner as in Test Example 1 are shown in FIG. 34. In FIG. 34, the results of Example 9 are shown together with the results of Example 1. As shown in FIG. 34, the tetramer (the peak indicated by arrow in the drawing) of the by-product confirmed in Example 1 was not detected in Example 9.

### Test Example 5: Design and Production of Multispecific Antibodies (Anti-CD20×CD3 Bispecific Antibody and Anti-BCMA×CD3 Bispecific Antibody)

(1) Design of Bispecific Antibody

(1-1) Design of Anti-CD20×CD3 Bispecific Antibody (CD20×CD3 TribsMab CLC)

A bispecific antibody corresponding to the multispecific antibody 12 of FIG. 5 was designed. The bispecific antibody was designed to target CD20 and CD3. As for the variable regions (portions surrounded by dashed lines in FIG. 5), the same sequences as those of the variable regions of REGN1979 (Eric J. Smith, Kara Olson, Lauric J. Haber, Bindu Varghese, Paurene Duramad. Sci Rep, 5, 17943 (2016)) known as the anti-CD20×CD3 bispecific antibody, that is, sequences of the heavy-chain variable region 1979VH-CD20 (specific to CD20) and the heavy-chain variable region 1979VH-CD3 (specific to CD3) of REGN1979, and the light-chain variable region 1979VL of REGN1979 were adopted. The light chain class of REGN1979 is λ. As for the constant region, a sequence derived from the human IgG1 class was adopted. As a sequence corresponding to the peptide linker LL of FIG. 5, a peptide linker having GGGGS as a basic sequence was designed.

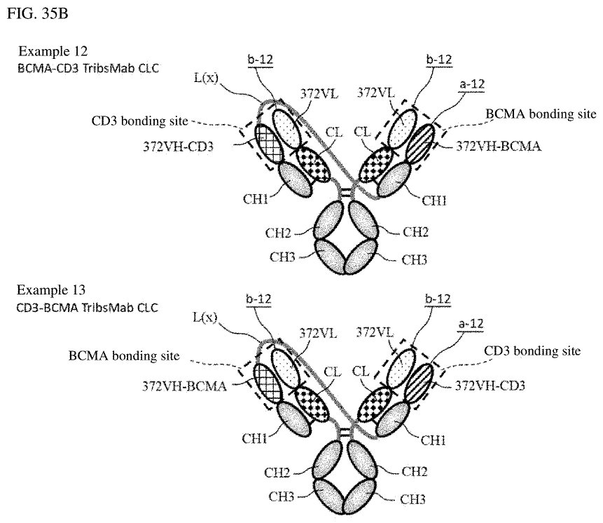

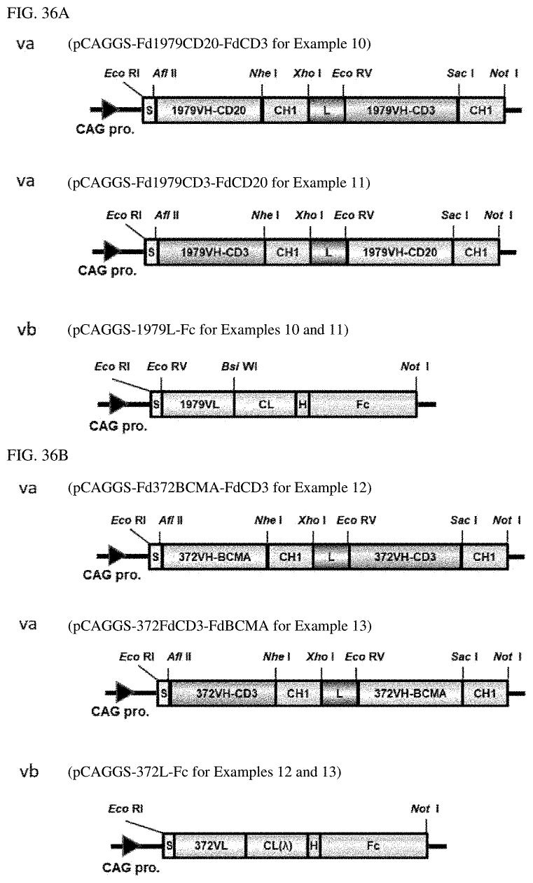

The correspondence relationship between the domains of the multispecific antibody 12 of FIG. 5 and the designed anti-CD20×CD3 bispecific antibody is shown in the following Tables 7 and 8, and the schematic diagram of the designed anti-CD20×CD3 bispecific antibody is shown in FIG. 35A.

TABLE 7

| | a-12 chain | | | | |
|---|---|---|---|---|---|
| | VHa1 | CHa1 | LL | VHa2 | CHa2 |
| Example 10 | 1979VH- | Human | L (70, delP); ≈245 Å | 1979VH- | Human |
| (CD20 - CD3 | CD20 | IgG1 CH1 | S2(G4S)13(G2S) | CD3 | IgG1 CH1 |
| TribsMab CLC) | | | | | |
| Example 11 | 1979VH- | | | 1979VH- | |
| (CD3 - CD20 | CD3 | | | CD20 | |
| TribsMab CLC) | | | | | |

TABLE 8

b-12 chain

| | VLb | CLb | CH2 | CH3 |
|---|---|---|---|---|
| Examples | 1979VL | Human | Human | Human |
| 10 and 11 | | IgG1 CH1 | IgG1 CH2 | IgG1 CH3 |

(1-2) Design of Anti-BCMA×CD3 Bispecific Antibody (BCMA×CD3 TribsMab CLC)

A bispecific antibody corresponding to the multispecific antibody 12 of FIG. 5 was designed. The bispecific antibody was designed to target BCMA and CD3. As for the variable regions (portions surrounded by dashed lines in FIG. 5), the same sequences as those of the variable regions of pSCHLI372 (Japanese Patent Laid-open Publication No. 2018-502062) known as the anti-BCMA×CD3 bispecific antibody, that is, sequences of the heavy-chain variable region 372VH-BCMA (specific to BCMA) and the heavy-chain variable region 372VH-CD3 (specific to CD3) of pSCHLI372, and the light-chain variable region 372VL of pSCHLI372 were adopted. The light chain class of pSCHLI372 is K. As for the constant region, a sequence derived from the human IgG1 class was adopted. As a sequence corresponding to the peptide linker LL of FIG. 5, a peptide linker having GGGGS as a basic sequence was designed.

The correspondence relationship between the domains of the multispecific antibody 12 of FIG. 5 and the designed anti-BCMA×CD3 bispecific antibody is shown in the following Tables 9 and 10, and the schematic diagram of the designed anti-BCMA×CD3 bispecific antibody is shown in FIG. 35B.

TABLE 9

| | a-12 chain | | | | |
|---|---|---|---|---|---|
| | VHa1 | CHa1 | LL | VHa2 | CHa2 |
| Example 12 | 372VH- | Human | L (70, delP); ≈245 Å | 372VH- | Human |
| (BCMA - CD3 | BCMA | IgG1 CH1 | S2(G4S)13(G2S) | CD3 | IgG1 CH1 |
| TribsMab CLC) | | | | | |
| Example 13 | 372VH- | | | 372VH- | |
| (CD3 - BCMA | CD3 | | | BCMA | |
| TribsMab CLC) | | | | | |

TABLE 10

b-12 chain

| | VLb | CLb | CH2 | CH3 |
|---|---|---|---|---|
| Examples | 372VL | Human | Human | Human |
| 12 and 13 | | IgG1 CL | IgG1 CH2 | IgG1 CH3 |

(2) Construction of Recombinant Vector for Bispecific Antibody Expression

The schematic diagrams of a recombinant vector va for a-12 chain expression and a recombinant vector vb for b-12 chain expression for expressing each of the bispecific antibodies designed in the above (1) are shown in FIGS. 36A and 36B, respectively. In the drawing, “S” represents a signal sequence, and “H” represents a hinge site.

First, the recombinant vectors va for a-12 chain expression (pCAGGS-Fd1979CD20-FdCD3, pCAGGS-Fd1979CD3-FdCD20, pCAGGS-Fd372BCMA-FdCD3, and pCAGGS-372FdCD3-FdBCMA) were prepared. The totally synthesized pEX-A2J2-1979VH-CD20, pEX-A2J2-1979VH-CD3, pEX-A2J2-372VH-BCMA, and pEX-A2J2-372VH-CD3 were digested with restriction enzymes Afl II and Nhe I and ligated to pCAGGS-Fd3958-Fd3178 digested with the same enzymes in advance to prepare four kinds of a-chain expression vector intermediates (pCAGGS-Fd1979CD20-Fd3178, pCAGGS-Fd1979CD3-Fd3178, pCAGGS-Fd372BCMA-Fd3178, and pCAGGS-Fd372CD3-Fd3178). Subsequently, four kinds of PCR products (Eco RV-1979VH-CD20-Sac I, Eco RV-1979VH-CD3-Sac I, Eco RV-372VH-BCMA-Sac I, and Eco RV-372VH-CD3-Sac I) were obtained by polymerase chain reaction (PCR) using pEX-A2J2-1979VH-CD20, pEX-A2J2-1979VH-CD3-1979VL, pEX-A2J2-372VH-BCMA, and pEX-A2J2-372VH-CD3 as templates, respectively. These four kinds of PCR products were digested with restriction enzymes Eco RV and Sac I and ligated to four kinds of a-chain expression vector intermediates digested with the same enzymes in advance to prepare pCAGGS-Fd1979CD20-FdCD3, pCAGGS-Fd1979CD3-FdCD20, pCAGGS-Fd372BCMA-FdCD3, and pCAGGS-372FdCD3-FdBCMA.

Subsequently, the recombinant vectors vb for b-12 chain expression (pCAGGS-1979L-Fc and pCAGGS-372L-Fc) were prepared. pEX-A2J2-1979VH-CD3-1979VL was digested with restriction enzymes Afl II and Nhe I and ligated to pCAGGS-128L-Fc digested with the same enzymes in advance to prepare pCAGGS-1979L-FC. Next, three kinds of PCR products (EcoRI-372VL, CLQ), and H-Fc-NotI) were amplified by PCR using pCAGGS-372-CH1-Fc, pCAGGS-HuM291LCQ), and pCAGGS-128L-Fc as templates, respectively, and these three kinds of PCR products were ligated by overlap extension to amplify EcoRI-372VL-CL(λ)-H-Fc-NotI. The final PCR product was digested with restriction enzymes Eco RI and Not I and ligated to pCAGGS-128L-Fc digested with the same enzymes in advance to prepare pCAGGS-372L-Fc.

(3) Preparation of Transformant

Expi293F cells derived from human embryonic kidney were used as a host. All the cultures were performed under the conditions of 37° C., 5% CO2, and 125 rpm.

The cells were cultured in an Erlenmeyer flask containing 30 mL of HE200 medium (Gmep), and transfection was performed when the number of cells reached 3-5×10⁶ cells/mL and the survival rate was 95% or more. For transfection, a solution obtained by adding 60 μg of plasmid (a-chain expression vector; 30 μg, b-chain expression vector; 30 μg) to 3 mL of Opti-MEM® (Thermo) in advance and a solution obtained by adding 240 μL of PEI-MAX (Polysciences) at 1 mg/mL to 3 mL of Opti-MEM® were prepared, respectively. These two solutions were mixed and left to stand still for 20 minutes, and then added to 54 mL of HE400 (Gmep), which was used as a transfection solution. The cell culture solution was dispensed into a 50 mL sample tube so that the number of cells was 150×10⁶ cells, centrifugation was performed at 1,500 rpm for 5 minutes, the culture solution was then removed, and the cells were suspended in the transfection solution and cultured for 20 hours.

(4) Production of Bispecific Antibody

Further, 150 μL of a 0.5 M sodium valproate aqueous solution and 240 μL of a 1 M sodium propionate aqueous solution were added, and the mixture was cultured for 6 days. The culture conditions are as described in the above (3).

(5) Purification of Bispecific Antibody

(5-1) Protein A Affinity Purification

Cells were removed from the culture solution by a centrifugation operation at 4° C. and at 6,000 rpm for 10 minutes, and the culture supernatant was filtered using a filter (Millex-HP, Millipore) having a pore size of 0.45 μm. The culture supernatant was added to KANEKA Kan CapA™ (column volume: 400 μL) pre-equilibrated with TBS (25 mM Tris, 150 mM NaCl, pH 7.2), followed by washing with TBS and elution with Gentle Ag/Ab Elution Buffer (Thermo). Each fraction in the purification process was analyzed by SDS-PAGE. The eluted fraction containing the target protein was first dialyzed against TBS, and then dialyzed twice against PBS (10 mM Na₂PO₄, 1.76 mM KH₂PO₄, 137 mM NaCl, 2.7 mM KCl, pH 7.4). The absorbance at 280 nm was measured using an absorptiometer (NanoPhotometer NP80, IMPLEN), and the target protein was quantified using an absorbance coefficient (0.1%, 280 nm) (BCMA×CD3 TribsMab CLC; 1.539 mg⁻¹·mL·cm⁻¹, CD20×CD3 TribsMab CLC; 1.471 mg⁻¹·mL·cm⁻¹) calculated from the amino acid sequence of TribsMab CLC. At that time, the absorbance at 320 nm was subtracted as a background. Each TribsMab CLC solution after protein A purification was added to a gel filtration chromatography column Superdex 200 10/300GL (GE Healthcare) equilibrated with PBS. Elution was performed using AKTA Prime plus (GE Healthcare) under the condition of a flow rate of 0.5 mL/min. Among the eluted fractions, each fraction including an absorption peak at 280 nm was obtained.

(5-2) Purification by Cation Exchange Chromatography

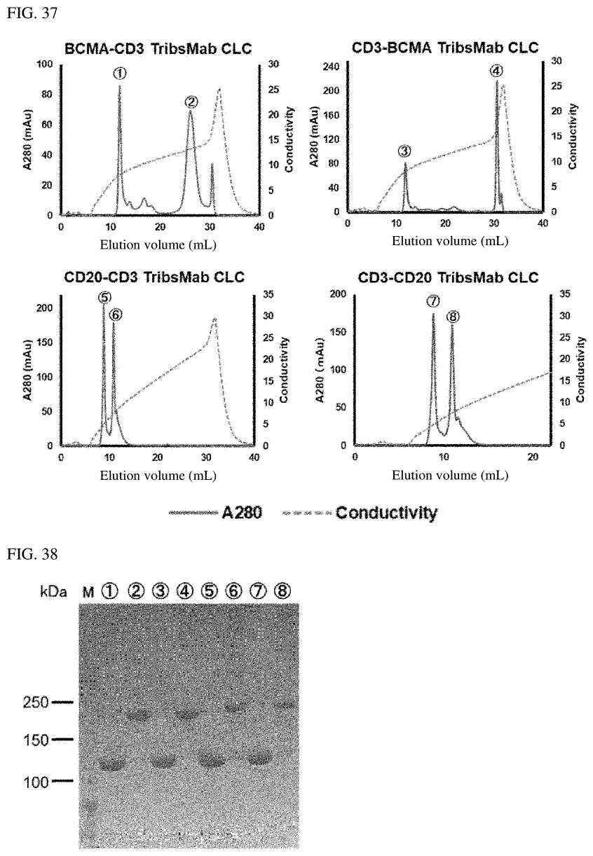

The fractions obtained in the above 5-1 were subjected to purification by cation exchange chromatography in the same manner as in 5-2 of Test Example 1. The cation exchange chromatograms for CD20×CD3 TribsMab CLC (Examples 10 and 11) and BCMA×CD3 TribsMab CLC (Examples 12 and 13) are shown in FIG. 37, and the analysis results of the obtained peak fractions by electrophoresis are shown in FIG. 38. As shown in FIG. 37, two peaks were confirmed in each of the fractions obtained in the above 5-1, and as shown in FIG. 38, it was found that the two peaks were the target bispecific antibody (band assigned with an even number in the drawing) and a b-chain by-product (band assigned with an odd number in the drawing), and it was found that these fractions can be separated.

From the above results and the results of 5-2 (FIG. 27) of Test Example 1, it became clear that the bispecific antibody of the present invention can separate the b-chain tetramer by cation exchange chromatography regardless of the variable region to be used.

### Test Example 6: Activity Evaluation of Multispecific Antibodies (Anti-CD20×CD3 Bispecific Antibody and Anti-BCMA×CD3 Bispecific Antibody)

(1) Binding Activity Evaluation of Anti-CD20×CD3 Bispecific Antibody

For the anti-CD20×CD3 bispecific antibodies of Examples 10 and 11 prepared in Test Example 5 (purified by cation exchange chromatography), the binding activity to Raji cells (CD20-positive cells) and T-LAK cells (CD3-positive cells) was evaluated as follows.

Raji cells cultured in a 10% FBS/RPMI 1640 medium and T-LAK cells cultured in a medium obtained by adding 1.4 μL of IL-2 (250 IU/μL) to 5 mL of 10% FBS/RPMI-1640 were used. As a primary antibody, a solution obtained by diluting each TribsMab CLC of Examples 10 and 11 with 0.1% NaN₃/PBS so as to have a final concentration of 500 nM was used. After CD20×CD3 TribsMab CLC and CD3×CD20 TribsMab CLC of Examples 10 and 11 were reacted with Raji cells or T-LAK cells, 1 μL of an anti-human IgG (Fc-specific)-FITC antibody (Sigma Aldrich) as a secondary antibody and 499 μL of 0.1% NaN₃/PBS were added and reacted. Analysis was performed using BD Accuri™ C6 (BD Biosciences).

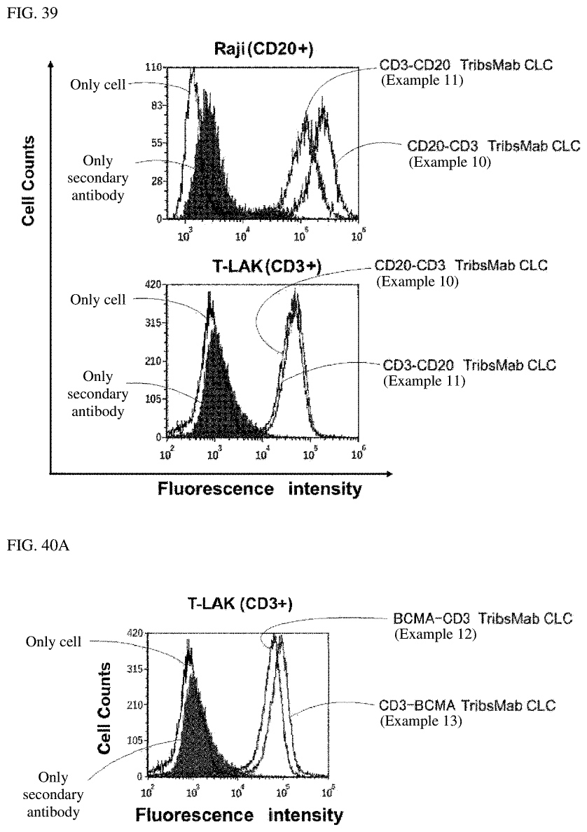

Results are shown in FIG. 39. As shown in FIG. 39, in CD20×CD3 TribsMab CLC of Examples 10 and 11, since the peak shift could be confirmed in both of the Raji cells (CD20-positive cells) and the T-LAK cells (CD3-positive cells), the binding activity to both of CD20 and CD3 was recognized.

(2) Binding Activity Evaluation of Anti-BCMA×CD3 Bispecific Antibody

For the anti-BCMA×CD3 bispecific antibodies of Examples 12 and 13 prepared in Test Example 5 (purified by cation exchange chromatography), the binding activity to T-LAK cells (CD3-positive cells) was evaluated as follows.

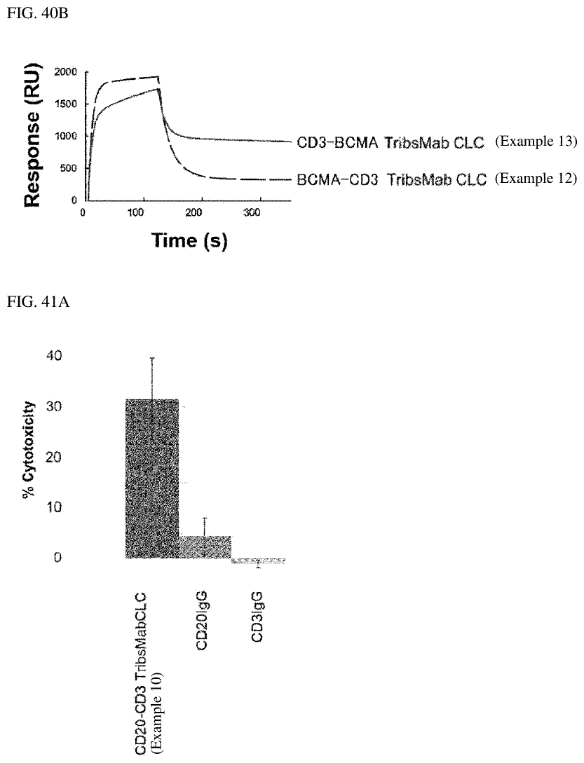

Raji cells cultured in a 10% FBS/RPMI 1640 medium and T-LAK cells cultured in a medium obtained by adding 1.4 μL of IL-2 (250 IU/μL) to 5 mL of 10% FBS/RPMI-1640 were used. As a primary antibody, a solution obtained by diluting each TribsMab CLC of Examples 10 and 11 with 0.1% NaN₃/PBS so as to have a final concentration of 500 nM was used. After BCMA×CD3 TribsMab CLC of Examples 12 and 13 was reacted with T-LAK, 1 μL of an anti-human IgG (Fc-specific)-FITC antibody (Sigma Aldrich) as a secondary antibody and 499 μL of 0.1% NaN₃/PBS were added and reacted. Analysis was performed using BD Accuri™ C6 (BD Biosciences). Results are shown in FIG. 40A. As shown in FIG. 40A, in BCMA×CD3 TribsMab CLC of Examples 12 and 13, since the peak shift could be confirmed in the T-LAK cells (CD3-positive cells), the binding activity to CD3 was recognized.

SPR analysis was performed using Biacore 3000 (GE Healthcare). The SPR analysis was performed using PBS (PBST) containing 0.005% Tween-20 as a running buffer solution under the condition of 25° C. First, BCMA-ECD-Fc was immobilized on the surface of a sensor chip CM5 using an amine coupling kit (GE Healthcare). Subsequently, in order to examine the specific binding between the antibody and the antigen, BCMA-CD3 TribsMab CLC and CD3-BCMA TribsMab CLC (1 μM each) were added onto the sensor chip at a flow rate of 20 μL/min for 2 minutes. As a regeneration reagent, 10 mM Glycine-HCl (pH 1.5) was used. Results are shown in FIG. 40B. As clearly shown from FIG. 40B, as a result of the interaction analysis between BCMA×CD3 TribsMab CLC of Examples 12 and 13 and BCMA by the SPR method, the response increased when each antibody was added onto the sensor chip on which BCMA-ECD-Fc was immobilized, and thus the binding activity to BCMA was recognized in these antibodies.

From the above, for BCMA×CD3 TribsMab CLC of Examples 12 and 13, the binding activity to both of BCMA and CD3 was recognized.

(3) Bispecificity Evaluation of Anti-CD20×CD3 Bispecific Antibody

The bispecificity of CD20×CD3 TribsMab CLC was evaluated by an LDH test using CD20-positive Raji cells (target cells) and CD3-positive T-LAK cells (effector cells). The experiment was performed under the condition of E (effector cell) T (target cell) ratio of 20:1. The T-LAK cells cultured in the same manner as in (1) of the present test example were suspended so as to be 4.0×10⁶ cells/mL, and then added in an amount of 25 μL per well. The Raji cells were suspended in 1% FBS/RPMI 1640 so as to be 2.0×10⁵ cells/mL, and then added in an amount of 50 μL per well. The antibody was diluted to a concentration of 40 nM, added in an amount of 25 μL per well, and then cultured for 3 hours. The operation after completion of the culture was performed according to the manual of Cytotoxicity LDH Assay Kit-WST (Dojindo Molecular Technologies). The absorbance of each well was measured using iMark™ Microplate Reader (BioRad). At that time, the absorbance at 630 nm was subtracted as a background, and the cytotoxicity rate was calculated from the following formula.

$$Cytotoxicity rate ( \% ) = ( \frac{\begin{matrix}{A}_{490} Sample Release - {A}_{490} effector SR - \\ {A}_{490} t arget SR\end{matrix}}{{A}_{490} t arget MR - {A}_{490} Target SR} ) ] \times 100 \qquad [ Mathematical Formula 2 ]$$

SR; Spontaneous Release Amount of LDH spontaneously released from cells
MR; Maximum Release Amount of LDH when 100% of cells in well are dissolved

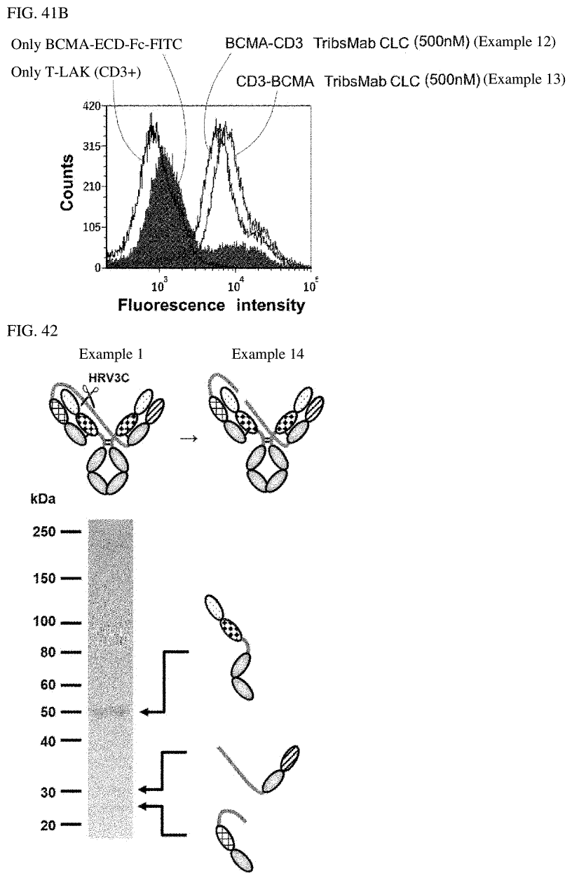

Results are shown in FIG. 41A. As clearly shown from FIG. 41A, since CD20-CD3 TribsMab CLC of Example 10 significantly induced cytotoxicity as compared with the monospecific antibody, it is considered that CD20-CD3 TribsMab CLC crosslinked the CD20-positive Raji cell and the CD3-positive T-LAK cell.

(4) Bispecificity Evaluation of Anti-BCMA×CD3 Bispecific Antibody

The bispecificity of BCMA×CD3 TribsMab CLC was evaluated by flow cytometric analysis using the CD3-positive T-LAK cells and BCMA-ECD-Fc-FITC. To the T-LAK cells cultured in the same manner as in (1) of the present test example, 500 nM of BCMA×CD3 TribsMab CLC was added as a primary antibody. Thereafter, BCMA-ECD-Fc-FITC was added so as to have a final concentration of 250 nM.

Results are shown in FIG. 41B. As clearly shown from FIG. 41B, it was found that BCMA×CD3 TribsMab CLC of Examples 12 and 13 crosslinked the fluorescently labeled BCMA-ECD-Fc and the CD3-positive T-LAK cell.

### Test Example 7: Bispecific Antibody with Linker Cleaved

The linker of HER2×HER3 TribsMab CLC of Example 1 was cleaved with an HRV3 protease to prepare Her2×HER3 TribsMab CLC (Example 14) in a linker-cleaved state.

To 1 mg of HER2×HER3 TribsMab CLC (after protein A purification) of Example 1, 5 μL of Turbo3C (HRV3C) protease (Funakoshi Co., Ltd.) was added, and the mixture was left to stand still at 4° C. overnight. Next, in order to remove the protease, purification was performed by column chromatography using Glutathione Sepharose 4B (GE Healthcare). After equilibration with PBS, each antibody solution was added and the flow-through was recovered. The remaining protease was eluted using an elution buffer (50 mM Tris-HCl, 10 mM reduced glutathione, pH 8.0) after the column was washed with PBS. The purified sample was analyzed by SDS-PAGE.

Results of SDS-PAGE are shown in FIG. 42. As clearly shown from FIG. 42, it could be confirmed that the linker of HER2×HER3 TribsMab CLC of Example 1 was cleaved, and the bispecific antibody of Example 14 was obtained.

The binding activity evaluation of the linker-cleaved HER2×HER3 TribsMab CLC of Example 14 obtained as described above by flow cytometry to HER2 and HER3-positive human mammary adenocarcinoma cells MCF-7 was performed.

Using MCF-7 cells cultured in a 10% FBS/DMEM medium, the linker-cleaved HER2×HER3 TribsMab CLC (500 nM) of Example 14 as a primary antibody was reacted with MCF-7 cells for 30 minutes, and then washed twice with 0.1% NaN₃/PBS. Subsequently, 1 μL of an anti-human IgG (Fc-specific)-FITC antibody (Sigma Aldrich) as a secondary antibody and 499 μL of 0.1% NaN₃/PBS were added and reacted for 30 minutes, and then washed twice with 0.1% NaN₃/PBS. Thereafter, the cells were subjected to flow cytometric analysis using BD Accuri™ C6 (BD Biosciences).

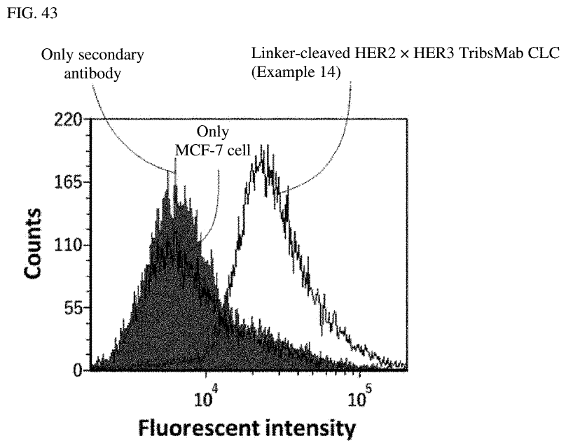

Results are shown in FIG. 43. As clearly shown from FIG. 43, the linker-cleaved HER2×HER3 TribsMab CLC of Example 14 also maintained binding activity.

### DESCRIPTION OF REFERENCE SIGNS

1, 1′, 11, 11′, 11″, 11′″, 12, 13, 21, 22, 23, 31, 32, 33: Multispecific antibody
a, a-11, a-12, a-13, a-21, a-22, a-23, a-31, a-2, a-33: Polypeptide a chain
Va1: Variable region Va1
VHa1: Heavy-chain variable region VHa1
Ca1: Constant region Ca1
CHa1: Heavy-chain constant region CHa1
LL, LL-11: Peptide linker LL
Lr1: Protease recognition sequence Lr1
Lr2: Protease recognition sequence Lr2
Va2: Variable region Va2
VHa2: Heavy-chain variable region VHa2
Ca2: Constant region Ca2
CHa2: Heavy-chain constant region CHa2
b, b-1′, b-12, b-13: Polypeptide b chain
Vb: Variable region Vb
VLb: Light-chain variable region VLb
Cb: Constant region Cb
CLb: Light-chain constant region CLb
ScFv1, ScFv2: Single-chain antibody
a′: Polypeptide a′ chain
a″: Polypeptide a″ chain
Lr1′: Cleavage fragment Lr1′ of protease recognition sequence Lr1
Lr2′: Cleavage fragment Lr2′ of protease recognition sequence Lr2
va: Recombinant vector va
vb: Recombinant vector vb
va′: Expression vector va′
vb′: Expression vector vb′

1. A multispecific antibody comprising a Fab region that includes one polypeptide a chain below and two polypeptide b chains below:
the polypeptide a chain including a polypeptide in which a variable region Va1, a constant region Ca1, a peptide linker LL, a variable region Va2, and a constant region Ca2 are linked in the stated order; and
the polypeptide b chain including a polypeptide in which a variable region Vb is linked to a constant region Cb binding to the constant region Ca1 or the constant region Ca2.

2. The multispecific antibody according to claim 1, wherein a length of the peptide linker LL is 70 to 280 Å.

3. The multispecific antibody according to claim 1, wherein the peptide linker LL includes a protease recognition sequence.

4. The multispecific antibody according to claim 3, wherein the peptide linker LL includes a protease recognition sequence Lr1 on the constant region Ca1 side and a protease recognition sequence Lr2 on the variable region Va2 side.

5. The multispecific antibody according to claim 1, wherein the multispecific antibody is IgD, IgE, IgG, or F(ab′)2.

6. The multispecific antibody according to claim 1, wherein the polypeptide a chain includes a polypeptide in which a heavy-chain variable region VHa1, a heavy-chain constant region CHa1, a peptide linker LL, a heavy-chain variable region VHa2, and a heavy-chain constant region CHa2 are linked in the stated order.

7. The multispecific antibody according to claim 1, wherein the polypeptide a chain includes a polypeptide in which a heavy-chain variable region VHa1, a light-chain constant region CLa1, a peptide linker LL, a heavy-chain variable region VHa2, and a light-chain constant region CLa2 are linked in the stated order.

8. The multispecific antibody according to claim 1, wherein a single-chain antibody further binds to the variable region Va1 and/or the constant region Ca2.

9. A multispecific antibody comprising a Fab region including one polypeptide a′ chain below, one polypeptide a″ chain below, and two polypeptide b chains below:
the polypeptide a′ chain including a polypeptide in which a variable region Va1, a constant region Ca1, and a cleavage fragment Lr1′ of a protease recognition sequence Lr1 are linked in the stated order;
the polypeptide a″ chain including a polypeptide in which a cleavage fragment Lr2′ of a protease recognition sequence Lr2, a variable region Va2, and a constant region Ca2 are linked in the stated order; and
the polypeptide b chain including a polypeptide in which a variable region Vb is linked to a constant region Cb binding to the constant region Ca1 or the constant region Ca2.

10. A DNA encoding a polypeptide a chain including a polypeptide in which a variable region Va1, a constant region Ca1, a peptide linker LL, a variable region Va2, and a constant region Ca2 are linked in the stated order.

11. A transformant obtained by transforming a host with:
a recombinant vector va that includes a DNA according to claim 10; and
a recombinant vector vb that includes a DNA encoding a polypeptide b chain including a polypeptide in which a variable region Vb is linked to a constant region Cb binding to the constant region Ca1 or the constant region Ca2.

12. A method for producing a multispecific antibody according to claim 1, comprising an antibody production step of culturing a transformant, the transformant being obtained by transforming a host with:
a recombinant vector va that includes a DNA encoding a polypeptide a chain including a polypeptide in which a variable region Va1, a constant region Ca1, a peptide linker LL, a variable region Va2, and a constant region Ca2 are linked in the stated order; and
a recombinant vector vb that includes a DNA encoding a polypeptide b chain including a polypeptide in which a variable region Vb is linked to a constant region Cb binding to the constant region Ca1 or the constant region Ca2.

13. The method for producing a multispecific antibody according to claim 12, wherein the peptide linker LL includes a protease recognition sequence Lr1 on the constant region Ca1 side and a protease recognition sequence Lr2 on the variable region Va2 side, and wherein the method further comprises a linker cleavage step of cleaving the peptide linker LL in the produced antibody by using proteases corresponding to the protease recognition sequence Lr1 and the protease recognition sequence Lr2 after the antibody production step.

14. A multispecific antibody preparation kit, comprising:
an expression vector va′ including a cloning site CS1 for incorporating a variable region Va1, a DNA encoding a constant region Ca1, a DNA encoding a peptide linker LL, a cloning site CS2 for incorporating a variable region Va2, and a DNA encoding a constant region Ca2 in the stated order; and
an expression vector vb′ including a cloning site CS for incorporating a variable region Vb, and a DNA encoding a constant region Cb binding to the constant region Ca1 or the constant region Ca2.

15. A diagnostic agent comprising the multispecific antibody according to claim 1.

16. A pharmaceutical composition comprising the multispecific antibody according to claim 1.
